(19) United States
(12) Patent Application Publication
(10) Pub. No.: US 2014/0045265 A1
(43) Pub. Date: Feb. 13, 2014

(54) ROBUST AND EFFICIENT DIFFERENTIATION OF HUMAN PLURIPOTENT STEM CELLS TO MULTIPOTENT VASCULAR PROGENITORS

(71) Applicants: Juan Carlos Izpisua Belmonte, La Jolla, CA (US); Ignacio Sancho-Martinez, La Jolla, CA (US); Leo Kurian, La Jolla, CA (US); Emmanuel Nivet, La Jolla, CA (US)

(72) Inventors: Juan Carlos Izpisua Belmonte, La Jolla, CA (US); Ignacio Sancho-Martinez, La Jolla, CA (US); Leo Kurian, La Jolla, CA (US); Emmanuel Nivet, La Jolla, CA (US)

(73) Assignee: Salk Institute For Biological Studies, La Jolla, CA (US)

(21) Appl. No.: 13/985,852

(22) PCT Filed: Feb. 16, 2012
(86) PCT No.: PCT/US2012/025449

§ 371 Date: Oct. 22, 2013

Related U.S. Application Data
(60) Provisional application No. 61/443,610, filed on Feb. 16, 2011.

(51) Int. Cl. C12N 5/077; C12N 5/078; C12N 5/071

(52) U.S. Cl. CPC C12N 5/0661; C12N 5/0602; C12N 5/0634
USPC 435/377

(57) ABSTRACT

Provided herein are methods and compositions for generating vascular progenitor cells that can in turn be differentiated into endothelial, smooth muscle, and hematopoietic cells. The present methods provide a faster, safer, and more efficient in vitro differentiation program than existing methodologies. The differentiated cells can be used for research and therapeutic applications, such as cell transplantation.

### CROSS-REFERENCES TO RELATED APPLICATIONS

This disclosure claims priority to U.S. Provisional Application No. 61/443,610, filed Feb. 16, 2011, the disclosure of which is incorporated by reference in its entirety.

### BACKGROUND OF THE INVENTION

In the developing embryo, mesoderm-derived multipotent progenitors, namely hemangioblasts, are the founders of both the hematopoietic and vascular systems (Choi et al. (1998) Development 125:725). Hemangioblasts are first observed in the blood islands from E7.5 in a developing mouse embryo and have primitive hematopoietic and vascular differentiation potential (Huber et al. (2004) Nature 432:625). However, hemangioblasts do not show long-term multilineage hematopoietic repopulation of an irradiated immunodeficient recipient (Cumano et al. (2001) Immunity 15:477). Definitive hematopoiesis takes place in mice by E10.5 in the aorta-gonad-mesonephros (AGM) and vitelline and umbilical arteries (Orkin et al. (2002) Nat Immunol 3:323). At this point, Hematopoietic Stem/Progenitor Cells (HSC/HPCs) are derived from endothelial cells with hemogenic capacity, i.e., the hemogenic endothelium. In humans, primitive hematopoiesis takes place by week 3 of embryonic development in the developing blood vessels of the yolk sac and subsequently from the aorta and vitelline artery's walls (Wang et al. 2004 Immunity 21:31). These progenitors are characterized by the expression of both hematopoietic and endothelial cell surface markers, thus defining an intermediate population with multilineage potential. Although the same ontology of markers cannot be directly extrapolated to humans, the generation of human hemangioblast-like cells with multilineage differentiation potential has been described (Dzierzak et al. (2008) Nat Immunol 9:129; Jaffredo et al. (2005) Exp Hematol 33:1029; Lancrin et al. (2009) Nature 457:892).

Multipotent vascular progenitor cells hold enormous promise for cell replacement therapies. Unfortunately, current methods for generating human multipotent progenitor cells with hemangioblast-like characteristics have shown low efficiency of differentiation, limited expansion potential, use of feeder layers, and relatively long differentiation times (Choi et al. (2009) Stem Cells 27:559; Grigoriadis et al. (2010) Blood 115:2769; Levenberg et al. (2010) Nat Protoc 5:1115; Park et al. (2010) Blood; Vodyanik et al. (2005) Blood 105:617; Lu et al. (2007) Nat Methods 4:501). Each of these factors hamper the use of these cells in the clinic (Peters et al. (2010) Int J Dev Biol 54:965). Moreover, these cell have shown early senescence and apoptosis (Feng et al. (2010) Stem Cells 28:704).

The present invention provides a robust method for the efficient, directed differentiation of human embryonic stem (hES) cells and human induced pluripotent stem cells (hiPS) into hemangioblast-like cells (i.e., vascular progenitor cells). Within 4 days of differentiation, the critical transcription factors for generation of vascular progenitor cells were activated, faithfully tracking key embryonic landmarks. Moreover, 30-65% multipotent progenitors could be generated from multiple hES/hiPS lines in only 8 days. The vascular progenitor cells were capable of efficient generation of functional, differentiated cells of the endothelial, smooth muscle, and hematopoietic lineages. The differentiated endothelial cells were able to contribute to the vasculature in vivo, indicating the applicability of the present methods for cell replacement therapies.

### BRIEF SUMMARY OF THE INVENTION

Provided herein are methods for generating a vascular progenitor cell that include culturing a pluripotent stem cell in a solution with BMP4, bFGF, or VEGF, thereby generating a vascular progenitor cell. The solution typically includes all three factors, and represents a chemically-defined, serum-free media. Thus, in some embodiments, the solution lacks feeder cells, serum, and activin A. In some embodiments, the pluripotent stem cell is derived from an embryonic stem cell, cord blood cell, adult stem cell, or induced pluripotent cell.

In some embodiments, the vascular progenitor cells are further differentiated. In some embodiments, vascular progenitor cells are separated from non-vascular progenitor cells prior to further differentiation. In some embodiments, the vascular progenitor cell is further differentiated into an endothelial cell, smooth muscle cell, or hematopoietic lineage cell.

Further provided are methods for generating an endothelial cell that include culturing a pluripotent stem cell in a solution comprising BMP4, bFGF, and VEGF to generate a vascular progenitor cell; and culturing the vascular progenitor cell in endothelial cell growth media to generate an endothelial cell. Isolated endothelial cells generated according to the disclosed methods are also provided herein.

In some aspects, provided herein are methods of treating an endothelial cell deficiency in a subject that include administering an endothelial cell generated according to the present methods to the subject, thereby treating the endothelial cell deficiency in the subject. In some embodiments, the methods include culturing a vascular progenitor cell in endothelial cell growth media to generate the endothelial cell for administration. In some embodiments, the methods include culturing a pluripotent stem cell in a solution comprising BMP4, bFGF, and VEGF to generate the vascular progenitor cell for further differentiation.

Provided herein are methods for generating a smooth muscle cell that include culturing a pluripotent stem cell in a solution comprising BMP4, bFGF, and VEGF to generate a vascular progenitor cell; and culturing the vascular progenitor cell in smooth muscle cell growth media to generate a smooth muscle cell. Isolated smooth muscle cells generated according to the disclosed methods are further provided herein.

Further provided herein are methods of treating a smooth muscle cell deficiency in a subject that include administering a smooth muscle cell generated according to the present methods to the subject, thereby treating the smooth muscle cell deficiency in the subject. In some embodiments, the methods include culturing a vascular progenitor cell in smooth muscle cell growth media to generate the smooth muscle cell for administration. In some embodiments, the methods include culturing a pluripotent stem cell in a solution comprising BMP4, bFGF, and VEGF, to generate the vascular progenitor cell for further differentiation.

Further provided are methods for generating a hematopoietic cell that include culturing a pluripotent stem cell in a solution comprising BMP4, bFGF, and VEGF to generate a vascular progenitor cell; and culturing the vascular progenitor cell in hematopoietic cell growth media to generate a hematopoietic cell. Isolated hematopoietic cells generated according to the disclosed methods are also provided herein.

In some aspects, provided herein are methods of treating a hematopoietic cell deficiency in a subject that include administering an endothelial cell generated according to the present methods to the subject, thereby treating the endothelial cell deficiency in the subject. In some embodiments, the methods include culturing a vascular progenitor cell in hematopoietic cell growth media to generate the hematopoietic cell for administration. In some embodiments, the methods include culturing a pluripotent stem cell in a solution comprising BMP4, bFGF, and VEGF to generate the vascular progenitor cell for further differentiation.

### BRIEF DESCRIPTION OF THE DRAWINGS

FIG. 1. Generation of multipotent progenitors follows embryonic developmental hallmarks. (a) Scheme and representative bright field pictures during the course of differentiation of human pluripotent stem cells towards CD34+ multipotent progenitors; (b) Directed differentiation leads to rapid downregulation of the stem cell-related marker CD 133 and concomitant upregulation of the hemangioblast-related marker CD34; c-f) Flow cytometry analysis of the hemangioblast-related markers CD34 and CD31 over the differentiation course of Hues9 embryonic stem cells (c), H1 embryonic stem cells (d), two-factor cord blood-derived iPS cells (CBiPS), (e) and four factor keratynocyte-derived iPS cells (KiPS) (f); (g) Representative flow cytometry plots depicting a double CD34+CD31+ population obtained after 8 days of differentiation. Upper panel shows isotype controls. Lower panel shows specific CD34 and CD31 staining; (h) and (i) mRNA expression profiling of pluripotency, mesodermal and hemangioblast-related markers upon differentiation of Hues9 embryonic stem cells (h) and KiPS (i).

FIG. 2. H1 ES derived multipotent progenitor generation follows embryonic developmental hallmarks. mRNA expression profiling of pluripotency, mesodermal and hemangioblast-related markers upon differentiation of H1 embryonic stem cells.

FIG. 3. CBiPS derived multipotent progenitor generation follows embryonic developmental hallmarks. mRNA expression profiling of pluripotency, mesodermal and hemangioblast-related markers upon differentiation of CBiPS cells.

FIG. 4. Detailed analysis of the different CD34+ populations obtained by day 8 of differentiation. (a) Representative Flow Cytometry gating strategy. Briefly, the living population was gated and displayed in terms of CD34 and CD31 expression on contour plots (upper panel). Each different population observed was further gated and analyzed in terms of their KDR and c-Kit expression (lower panels). All percentages described herein refer to the initial living population, representing the total number of cells after isotype background subtraction. (b) Percentages of positive cells for each different population observed in different ES and iPS lines. Note that most of the CD31+ cells are comprised inside a CD34+CD31+KDR+c-Kit+ quadruple positive population. The percentages of CD34+CD31− and CD34+CD31+ cells represent the total number of CD34+ cells, regardless their KDR and c-Kit expression, as represented in FIG. 1 to allow visual comparison. Detailed analysis of KDR and c-Kit expression of these populations is also shown.

FIG. 5. Hues9 derived CD34+ progenitors show hemangioblast differentiation potential. (a) Sorted CD34+ progenitors under endothelial differentiation conditions showed efficient and robust generation of endothelial-like cells as measured by flow cytometry analysis of VE-Cadherin and Endoglin expression. (b) Representative contour plots showing differentiated VE-Cadherin+Endoglin+ cells. (c)-(e) Fluorescence microscopy analysis showing the expression of indicated endothelial cell markers in Human embryonic stem 9 (Hues9) derived ECs. (f) mRNA expression profile showing specific upregulation of EC markers in Hues9 derived ECs. (g) Fluorescence microscopy analysis showing the expression of indicated SMC markers in Hues9-derived SMCs. (h) mRNA expression profile showing specific upregulation of SMC markers in Hues9-derived SMCs. (i)-(j) Hues9-derived ECs are functional in vitro. (i) Representative pictures of Acetylated-LDL (LDL) uptake by differentiated ECs (right panel) and the respective negative controls (left panel). (j) LDL Mean Fluorescence Intensities (MIF) of Hues9-derived endothelial cells compared to the respective negative control. (k) Hues9-derived ECs spontaneously formed capillary-like structures in matrigel. (l)-(p) Fifteen days after injection, matrigel plugs were extracted and subjected to analyses in 4 experimental groups (no cells, HUVEC (human umbilical vein endothelial cells), HUES9-derived endothelial and non endothelial cells). (1) Representative pictures showing the identification of human cells by in situ hybridization on ALU+ sequences (dark dot). (m) Representative pictures showing the identification of human endothelial cells by anti-human CD31 staining. (n) High magnification pictures showing expression of CD31 and vessel-like structure formation in Hues9. (o)-(p) Identification of human vessel-like structures by Uvex Lectin rhodamine staining in HUVEC (positive control) (o) and HUES9-derived endothelial cells (p) matrigel plugs.

FIG. 6. H1 derived CD34+ progenitors show angioblast differentiation potential. a-c) Fluorescence microscopy analysis showing the expression of indicated endothelial cell markers in H1-derived ECs; (d) mRNA expression profile showing specific upregulation of EC markers in H1-derived ECs; (e) Fluorescence microscopy analysis showing the expression of indicated SMC markers in H1-derived SMCs; (f) mRNA expression profile showing specific upregulation of SMC markers in H1-derived SMCs; (g) H1-derived endothelial cells show significantly higher uptake of acetylated LDL compared to the respective negative control. Quantification of the uptake of Acetylated LDL is represented as Mean Fluorescence Intensities (MIF) of the respective population. (h) H1-derived ECs spontaneously formed capillary-like structures in matrigel.

FIG. 7 KiPS-derived-CD34+ progenitors show angioblast differentiation potential. a-c) Fluorescence microscopy analysis showing the expression of indicated endothelial cell markers in KiPS-derived ECs (d) mRNA expression profile showing specific upregulation of EC markers in H1-derived ECs; (e) Fluorescence microscopy analysis showing the expression of indicated SMC markers in KiPS-derived SMCs; (f) mRNA expression profile showing specific upregulation of SMC markers in KiPS-derived SMCs; (g) KiPS-derived endothelial cells show significantly higher uptake of acetylated LDL compared to the respective negative control. Quantification of the uptake of Acetylated LDL is represented as Mean Fluorescence Intensities (MIF) of the respective population. (h) KiPS-derived ECs spontaneously formed capillary-like structures in matrigel.

FIG. 8. a-c) Fluorescence microscopy analysis showing the expression of indicated endothelial cell markers in CBiPS-derived ECs; (d) mRNA expression profile showing specific upregulation of EC markers in CBiPS-derived ECs; (e) Fluorescence microscopy analysis showing the expression of indicated SMC markers in KiPS-derived SMCs; (f) mRNA expression profile showing specific upregulation of SMC markers in CBiPS-derived SMCs; (g) CBPS-derived endothelial cells show significantly higher uptake of acetylated LDL compared to the respective negative control. Quantification of the uptake of Acetylated LDL is represented as Mean Fluorescence Intensities (MIF) of the respective population; (h) CBiPS-derived ECs spontaneously formed capillary-like structures in matrigel.

FIG. 9. Pluripotent Stem Cell-derived-CD34+ progenitors show hematopoietic differentiation potential. (a) Flow cytometry analysis of CD34 and the pan-hematopoietic related marker CD45 during the course of mesodermal differentiation; (b) Sorted CD34+ cells displayed restricted hematopoietic potential resembling primitive hematopoiesis when cultured in the presence of hematopoietic cytokines.

FIGS. 10A-10D: Generated endothelial cells demonstrate functionality in vivo. (A) Fifteen days post-injection, matrigel plugs were extracted for analysis. Increased blood circulation is evident through the extracted plugs, demonstrating connection with pre-existing vasculature (anastomosis). (B) Representative in situ hybridizations using Ulex Lectin-rhodamine shows vessel like structures; ALU+ sequences and anti-human CD31 show human cells; scale bars 5 um. Note circulating RBCs in vessels. (C) Fully differentiated endothelial cells did not give rise to teratoma formation as compared to undifferentiated iPSC controls (compare the huES9 and KiPS^{ENDO} images); scale bares 500 um. (D) For teratoma assays, the presence of human endothelial cells in injected tissue (testes) was confirmed with anti-human PECAM-1 immunostaining; scale bars 20 um.

### DETAILED DESCRIPTION OF THE INVENTION

The invention provides an efficient and robust method for the generation of CD34+ tripotent progenitor cells (i.e., hemangioblast-like cells, vascular progenitor cells). These tripotent progenitors follow a normal embryonic development pathway in terms of marker expression and differentiation potential. The tripotent progenitors efficiently differentiate into endothelial cells (ECs) and smooth muscle cells (SMCs), and give rise to the monocytic and erythroid lineages of hematopoietic cells. This pattern resembles primitive hematopoiesis as it occurs in the developing embryo (Oberlin et al. (2002) Development 129:4147).

The CD34+ tripotent progenitor cells can be generated in 8 days of in vitro differentiation, in the absence of embryoid body formation. The present methods also allow differentiation in the absence of Activin-A, previously believed to be indispensible for mesodermal differentiation. The system presented herein is well-defined, and is carried out in the absence of serum and feeder cells, thus providing single-step generation of tripotent progenitors in a monolayer culture. The present method simplifies culture conditions and cell handling, while reducing the potential presence and/or interference of feeder cell derived contaminants during differentiation, signaling studies and/or cell transplantation. The short time requirement and high efficiency provide the basis for the large-scale generation of tripotent progenitors necessary for clinical applications.

### I. DEFINITIONS

The terms “vascular progenitor cell,” “hemangioblast-like cell,” “tripotent progenitor cell,” and “mesodermal progenitor cell” refer to a cell generated from a pluripotent stem cell that can give rise to mesodermal lineage cell types, e.g., endothelial cells, smooth muscle cells, and/or hematopoietic cells. Vascular progenitor cells can be identified by expression of characteristic markers, e.g., CD34, and other vascular progenitor cell markers described herein.

A “pluripotent stem cell” is a cell that can self-renew through mitotic cell division and give rise to more differentiated cell types. Among mammalian stem cells, embryonic and somatic stem cells can be distinguished. Embryonic stem (ES) cells reside in the blastocyst and normally give rise to embryonic tissues, whereas somatic stem cells reside in adult tissues and are normally involved in tissue regeneration and repair. As used herein, pluripotent stem cells can be derived from ES cells or from induced pluripotent stem (iPS) cells, e.g., from adult tissues or umbilical cord blood. Human pluripotent stem cells typically express at least some of the markers selected from: SSEA-3, SSEA-4, TRA-1-60, TRA-1-81, TRA-2-49/6E, ALP, Sox2, E-cadherin, UTF-1, Oct4, Lin28, Rexl, and Nanog. Cell morphology of pluripotent stem cells can also be distinguished from other cell types.

“Self renewal” refers to the ability of a cell to divide and generate at least one daughter cell with the self-renewing characteristics of the parent cell. The second daughter cell may commit to a particular differentiation pathway. For example, a self-renewing hematopoietic stem cell can divide and form one daughter stem cell and another daughter cell committed to differentiation in the myeloid or lymphoid pathway. A committed progenitor cell has typically lost the self-renewal capacity, and upon cell division produces two daughter cells that display a more differentiated (i.e., restricted) phenotype. Non-self renewing cells refers to cells that undergo cell division to produce daughter cells, neither of which have the differentiation potential of the parent cell type, but instead generates differentiated daughter cells.

The terms “pluripotent,” “sternness,” “pluripotency,” etc. refer to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism.

“Allogeneic” refers to deriving from, originating in, or being members of the same species, where the members are genetically related or genetically unrelated but genetically similar. An “allogeneic transplant” refers to transfer of cells or organs from a donor to a recipient, where the recipient is the same species as the donor.

“Autologous” refers to deriving from or originating in the same subject or patient. An “autologous transplant” refers to collection and retransplant of a subject's own cells or organs.

The terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division. The term does not imply that all cells in the culture survive or grow or divide, as some may naturally senesce, etc. Cells are typically cultured in media, which can be changed during the course of the culture.

The terms “media” and “culture solution” refer to the cell culture milieu. Media is typically an isotonic solution, and can be liquid, gelatinous, or semi-solid, e.g., to provide a matrix for cell adhesion or support. Media, as used herein, can include the components for nutritional, chemical, and structural support necessary for culturing a cell.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

The term “isolated,” when referring to a cell or molecule (e.g., nucleic acid or protein), indicates that the cell or molecule is or has been separated from its natural environment. For example, an isolated cell can be removed from its host individual, but still exist in culture with other cells, or be reintroduced into its host individual.

The term “feeder-free,” refers to the absence of feeder cells. The term “feeder cell” is known in the art, and includes all cells used to support the propagation of stem cells, e.g., during the process of reprogramming. Feeder cells can be irradiated prior to being co-cultured with the stem cells in order to avoid the feeder cells outgrowing the stem cells. Feeder cells provide a layer physical support for attachment, and produce growth factors and extracellular matrix proteins that support cells. Examples of feeder cells include fibroblasts (e.g., embryonic fibroblasts), splenocytes, macrophages and thymocytes.

The terms “subject,” “patient,” “individual,” etc. are not intended to be limiting and can be generally interchanged. That is, an individual described as a “patient” does not necessarily have a given disease or deficiency, but may be merely seeking medical advice.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “dose” and “dosage” are used interchangeably herein. A dose refers to the amount of active ingredient (e.g., EC, SMC, or hematopoietic cell) given to an individual at each administration. For the present invention, the dose can be expressed, e.g., in cells/kg of the individual receiving the treatment, or cells/volume of the pharmaceutical solution administered.

The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical, and depends on the route of administration. For the present invention, the dosage form is typically in a liquid or semi-liquid form, such as a saline solution for injection.

As used herein, the terms “treat” and “prevent” are not intended to be absolute terms. Treatment can refer to any reduction in the frequency or severity of symptoms, amelioration of symptoms, improvement in patient comfort and/or function, etc. The effect of treatment can be compared to an individual or pool of individuals not receiving a given treatment, or to the same patient prior to, or after cessation of, treatment.

As used herein, the terms “pharmaceutically” acceptable is used synonymously with physiologically acceptable and pharmacologically acceptable. A pharmaceutical composition will generally comprise agents for buffering and preservation in storage, and can include buffers and carriers for appropriate delivery, depending on the route of administration.

The term “prevent” refers to a decrease in the occurrence of symptoms of a cell deficiency in a patient. As indicated above, the prevention may be complete (no detectable symptoms) or partial, such that fewer symptoms are observed than would likely occur absent treatment.

The term “therapeutically effective amount,” as used herein, refers to that amount of the therapeutic agent sufficient to ameliorate the disorder. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or “percent identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The sensitivity of the hybridization assays may be enhanced through use of a nucleic acid amplification system that multiplies the target nucleic acid being detected. Amplification can also be used for direct detection techniques. Examples of such systems include the polymerase chain reaction (PCR) system and the ligase chain reaction (LCR) system. Other methods include the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products in real time.

The words “protein”, “peptide”, and “polypeptide” are used interchangeably to denote an amino acid polymer or a set of two or more interacting or bound amino acid polymers.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

Technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989).

### II. PROGENITOR STEM CELLS

Progenitor stem cells for use in the present methods can be obtained from any source, e.g., primary cells from an individual or group of individuals, or from a cell line, e.g., an embryonic stem cell line. In some aspects, the progenitor stem cell is an induced pluripotent stem (iPS) cell. Methods for generating iPS cells are described, e.g., in Giorgetti et al. (2009) Cell Stem Cell 5:353-57; Gekas & Graf (2010) J Exp Med 207:2781; and Nelson et al. (2010) Stem Cells Cloning 3:29-37. Typically, generation of iPS cells involves exogenous expression or induction of at least one of c-Myc, SOX2, Oct3/4, and/or KLF.

In some aspects, the progenitor stem cells are derived from cord blood (e.g., from a cord blood bank), embryonic stem cells (e.g., from an approved line or from primary samples), or adult tissues such as bone marrow. In some aspects, the progenitor stem cell expresses cell surface markers that indicate pluripotent potential, e.g., CD133. Additional markers for pluripotency include SSEA-3, SSEA-4, TRA-1-60, TRA-1-81, TRA-2-49/6E, and Nanog.

In some aspects, the progenitor stem cells are obtained from the same individual that is intended to receive a transplant of differentiated cells generated by the progenitor stem cells. That is, the progenitor stem cells, and the differentiated cells generated therefrom, are autologous to the donor/recipient. In some aspects, the progenitor stem cell donor(s) is different than the recipient, such that the transplanted cells will be allogeneic to the recipient.

Progenitor stem cells can be from any mammal, e.g., human, non-human primate, mouse, rat, rabbit, dog, cat, horse, cow, pig, sheep, goat, etc. Typically the source of the cell is determined based on the intended use. For example, for transplant, progenitor stem cells from the same species can be used for differentiation into the cell type to be transplanted. In some aspects, however, a xenotransplant can be carried out such that progenitor stem cells from a different species are transplanted into the recipient.

### III. MESODERMAL, HEMANGIOBLAST, AND ANGIOBLAST CELLS

Provided herein are in vitro differentiated cells generated from pluripotent stem cells or vascular progenitor cells. These differentiated cells can be generated in vitro in a matter of days in the absence of potential contaminants, such as feeder cells and serum. The differentiated cells (e.g., endothelial cells, smooth muscle cells, erythrocytes, granulocyte-macrophage, and macrophage cells) can thus be used for transplant with a minimal risk of complications.

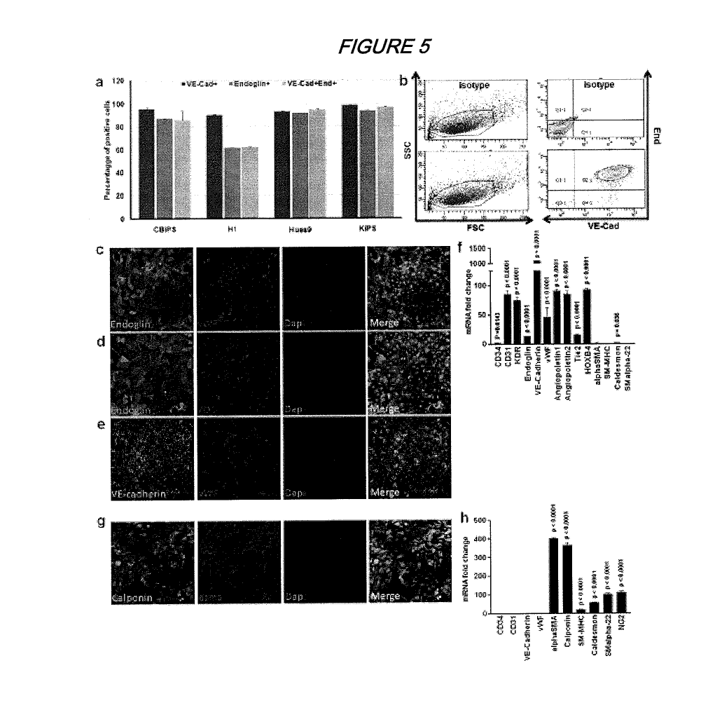

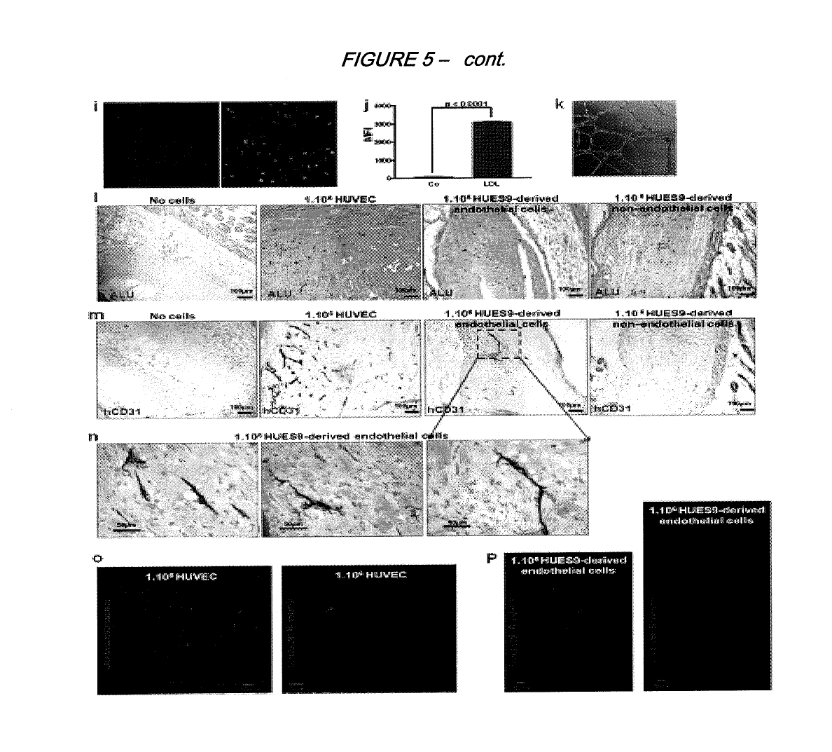

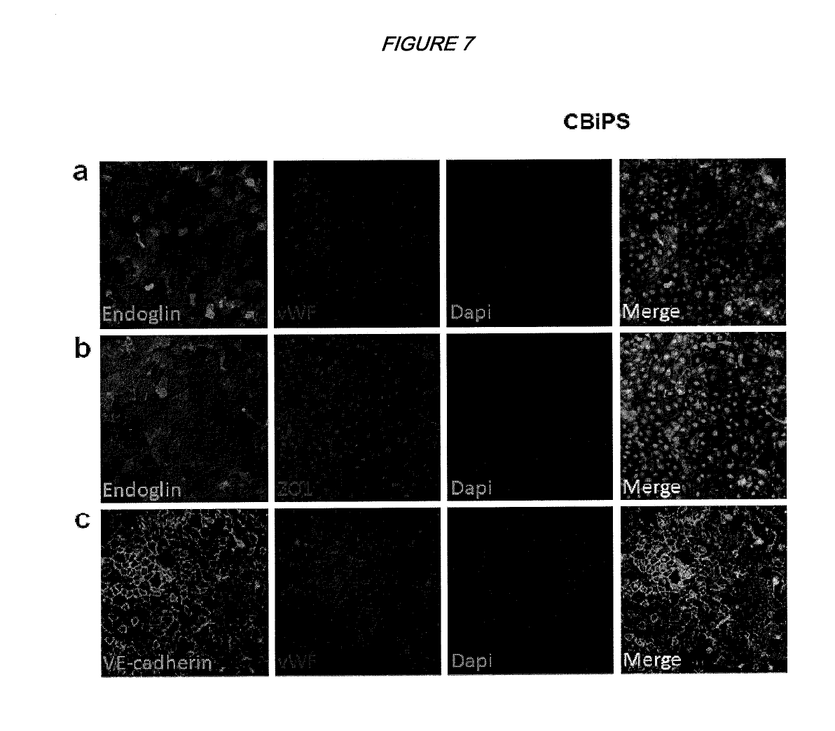

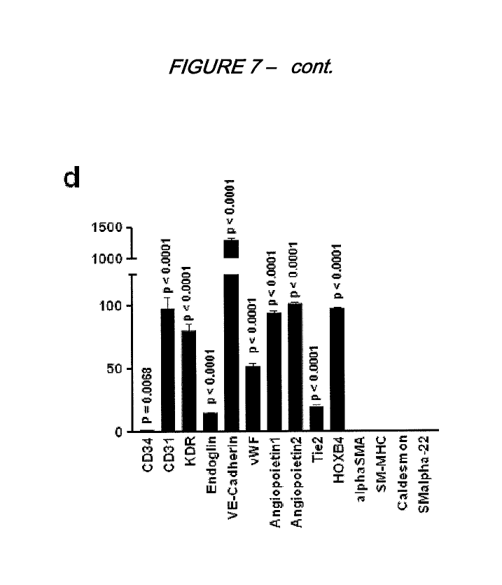

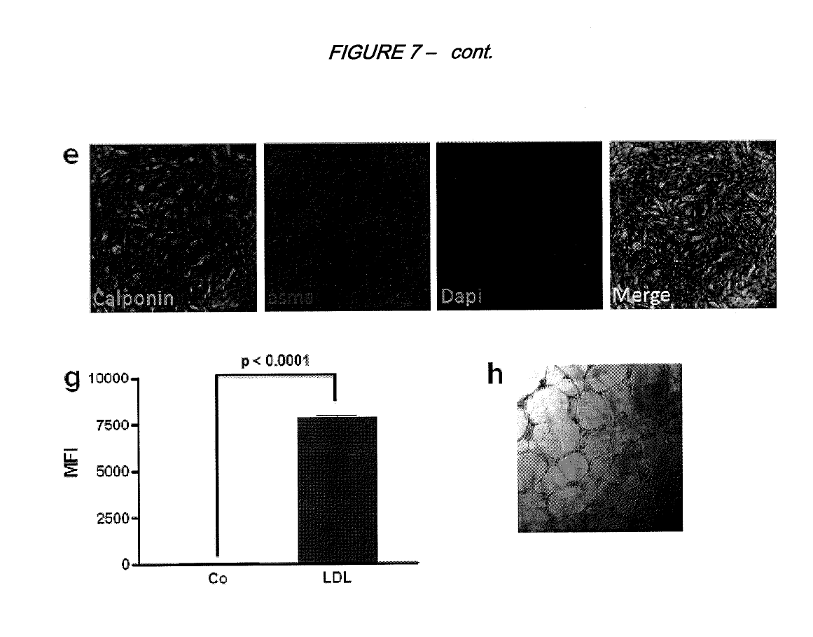

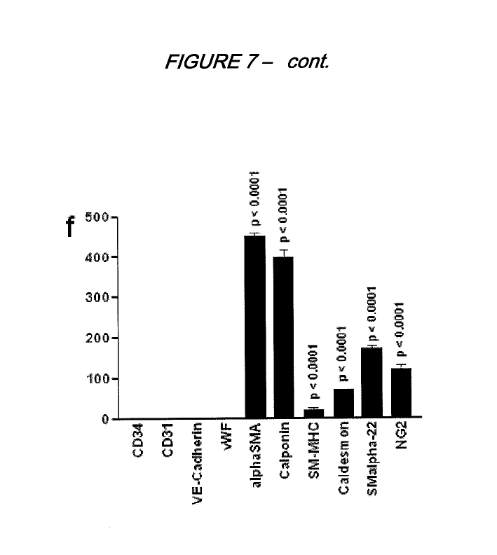

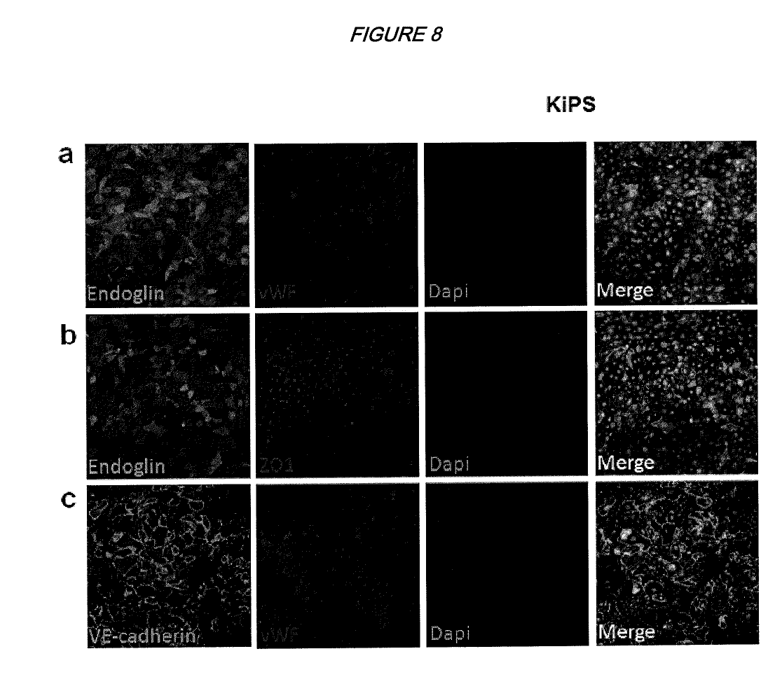

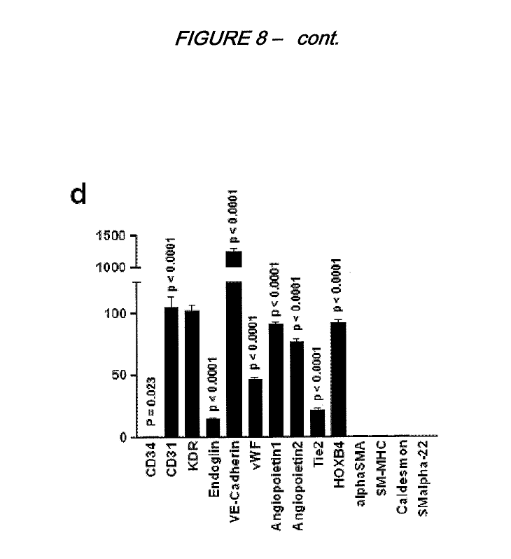

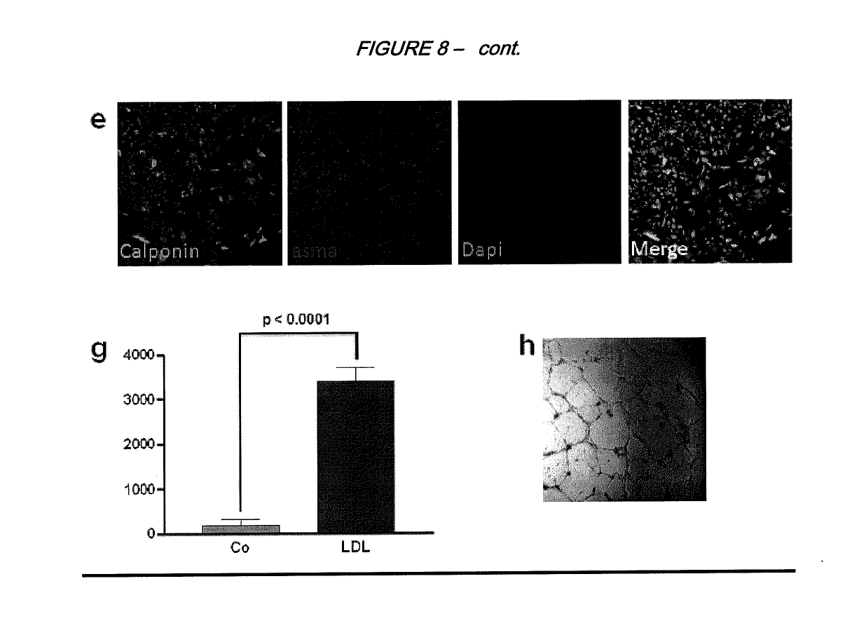

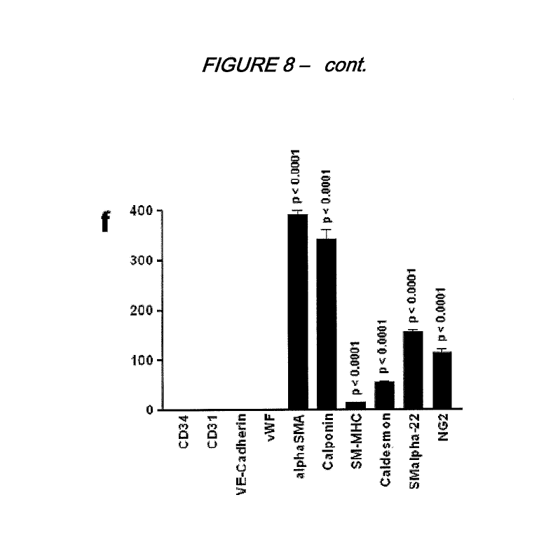

In some aspects, the in vitro differentiated cell is an isolated endothelial cell (EC). The isolated EC can be generated by culturing the vascular progenitor cell in endothelial cell growth media, thereby generating the EC. Thus, in some embodiments, the vascular progenitor cell is allowed to grow, divide, and/or differentiate, thereby generating the EC. In some aspects, the EC growth media includes EGF (endothelial growth factor), and optionally collagen. In some aspects, the vascular progenitor cell is CD34+. In some aspects, the EC expresses at least one EC marker protein, e.g., VE-cadherin, endoglin, vWF, Z01, angiopoietin1, and angiopoietin 2. In some aspects, the EC does not express non-EC markers, e.g., SMC markers. In some aspects, the EC is capable of vessel formation and/or acetylated LDL uptake. In some aspects, the endothelial cell can be identified morphologically (see, e.g., FIG. 5-8). In some aspects, the vascular progenitor cell is generated by culturing a pluripotent stem cell in a solution comprising BMP4, bFGF, and VEGF to generate the vascular progenitor cell for further differentiation. Details regarding culturing both vascular progenitor cells and pluripotent stem cells are described herein.

In some aspects, the in vitro differentiated cell is an isolated smooth muscle cell (SMC). The isolated SMC can be generated by culturing the vascular progenitor cell in SMC growth media, thereby generating the SMC. In some aspects, the SMC growth media includes at least one smooth muscle cell growth factor, and optionally collagen. In some aspects, the vascular progenitor cell is CD34+. In some aspects, the SMC expresses at least one SMC marker protein, e.g., calponin, alpha-smooth muscle actin, NG2, SMalpha-22, or caldesmon. In some aspects, the SMC does not express a non-SMC marker, e.g., an EC marker. In some aspects, the SMC can be identified morphologically (see, e.g., FIG. 5-8). In some aspects, the vascular progenitor cell is generated by culturing a pluripotent stem cell in a solution comprising BMP4, bFGF, and VEGF to generate the vascular progenitor cell for further differentiation.

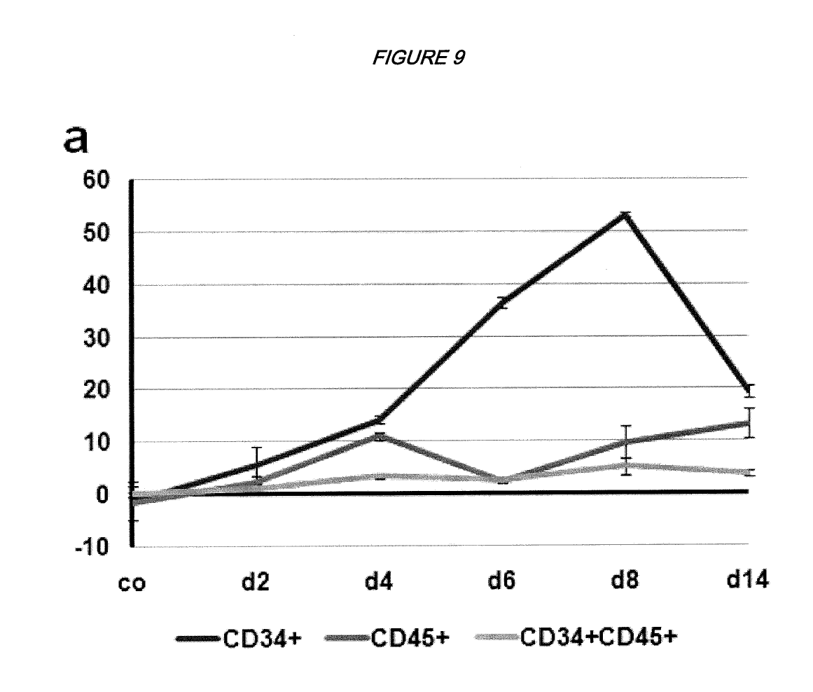

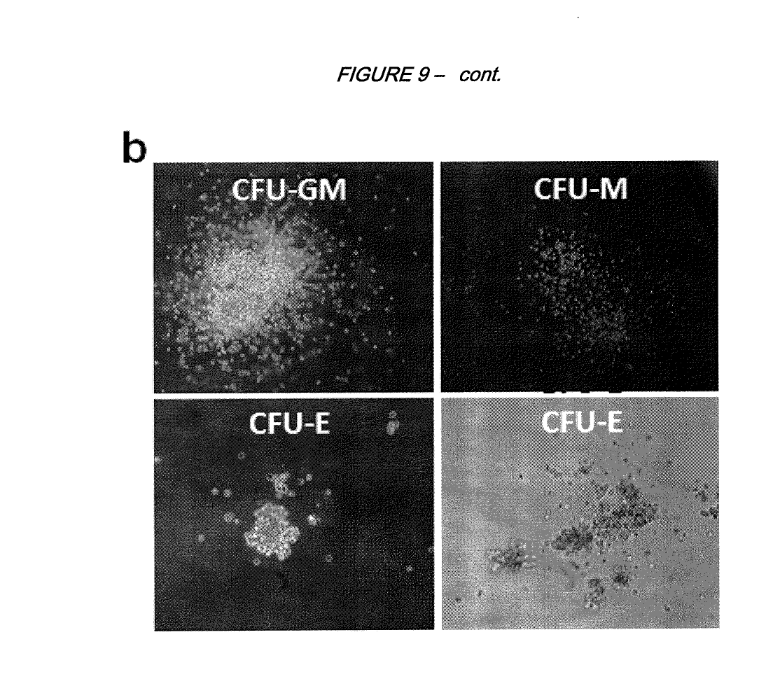

In some aspects, the in vitro differentiated cell is an isolated hematopoietic cell (e.g., erythrocyte, granulocyte-macrophage, or macrophage). The isolated hematopoietic cell can be generated by culturing the vascular progenitor cell in hematopoietic cell growth media, thereby generating the hematopoietic cell. In some aspects, the hematopoietic cell growth media includes at least one hematopoietic cell growth factor (e.g., stem cell factor (SCF), GM cell stimulating factor (GM-CSF), IL-3, EPO). The hematopoietic cell growth media can be semisolid, e.g., including methylcellulose or matrigel. In some aspects, the vascular progenitor cell is CD34+. In some aspects, the hematopoietic cell expresses at least one hematopoietic cell marker protein, e.g., CD45. In some aspects, the hematopoietic cell does not express a non-hematopoietic cell marker, e.g., an EC or SMC marker. In some aspects, the hematopoietic cell can be identified morphologically (see, e.g., FIG. 9), or determined based on colony forming activity. In some aspects, the vascular progenitor cell is generated by culturing a pluripotent stem cell in a solution comprising BMP4, bFGF, and VEGF to generate the vascular progenitor cell for further differentiation.

### IV. CELL CULTURE METHODS

Provided herein are methods for generating vascular progenitor cells that can include culturing (growing, maintaining) a pluripotent stem cell in a solution (e.g. media) comprising bone morphogenic protein 4 (BMP4), basic fibroblast growth factor (bFGF), and vascular endothelial growth factor (VEGF) to generate a vascular progenitor cell. In some aspects, the vascular progenitor cell is CD34+. In some aspects, the vascular progenitor also expresses at least one cell surface marker selected from CD31 (PECAM-1), c-Kit, and KDR. In some aspects, the vascular progenitor cell does not express CD 133. In some cases, the pluripotent stem cell is derived from an embryonic stem cell, a cord blood pluripotent cell, or an adult stem cell (e.g., an induced pluripotent stem cell (iPS)).

In some aspects, the culturing is carried out in the absence of feeder cells and/or Activin-A. In some aspects, the culturing is carried out in the absence of serum, that is, the culture solution (media) will lack serum.

In some aspects, the solution includes about 5-100 ng/ml BMP4, e.g., about 10-80, 20-50, 20-40, 10-50, 20-30, or about 25 mg/ml BMP4. In some aspects, the solution includes about 5-100 ng/ml bFGF, e.g., about 10-70, 10-50, 15-40, 15-25, or about 20 mg/ml bFGF. In some aspects, the solution includes about 5-200 ng/ml VEGF, e.g., 10-100, 20-80, 30-60, 40-60, 45-55, or about 50 ng/ml VEGF.

The methods of generating vascular progenitor cells can be carried out in less than 10 days of culture, but typically more than 2, 3, 4, 5, 6, or 7 days. In some aspects, the pluripotent stem cell is cultured for at least 4, 5, 6, 7, or 8 days, e.g., 6-9, 4-10, 5-10, 6-12, 7-12, 8-12, or 7-9 days. One of skill will understand that, during the culturing period, the initial pluripotent stem cell or population of pluripotent stem cells can divide and/or change character as the pluripotent stem cell gives rise to a vascular progenitor cell. Indeed, the method is typically carried out with a plurality of pluripotent stem cells to form a population of cells that includes, over time, increasing numbers of vascular progenitor cells. In some aspects, the population of cells comprises 30-80% vascular progenitor cells, e.g., 35, 40, 45, 50, 60, 65, 70, 75%, or higher percentage vascular progenitor cells.

In some cases, the vascular progenitor cells are separated from the population of cells, e.g., for further differentiation. Such separation can be carried out using any method known in the art, e.g., based on cell surface marker expression, cell size, or cell morphology.

Exemplary methods for cell separation include use of antibodies to cell surface markers (e.g., CD34, CD31, c-kit, and/or KDR for vascular progenitor cells) such as magnetic cell separation, sorting by flow cytometry, or chromatographic methods. For separation based on size, centrifugation, e.g., size exclusion centrifugation or density gradient centrifugation, can be used. See, e.g., Recktenwald (1998) Cell Separation Methods and Applications (Marcel Dekker ed.).

In some aspects, the invention provides methods for differentiation of vascular progenitor cells into, e.g., endothelial cells (ECs), smooth muscle cells (SMCs), or cells of hematopoietic lineage, e.g., erythroid, granulocyte-macrophage, or macrophage cell types. Such methods can be practiced in combination with the methods for generating a vascular progenitor cell, or separately, e.g., starting with a hemangioblast-like cell or vascular progenitor cell.

Methods for generating ECs include culturing a vascular progenitor cell in a solution appropriate for EC growth (e.g., EC growth media) and allowing the vascular progenitor cell to form an EC. In some aspects, the EC growth media includes endothelial cell growth factor, and the cells can optionally be grown in the presence of collagen. In some aspects, the vascular progenitor cell is cultured in EC growth media between 6 and 20 days, e.g., at least 6, 8, 10, 12 or 14 days (i.e., the course or duration of EC differentiation). In some aspects, the vascular progenitor cell is cultured in EC growth media for about 10-14 days. In some aspects, the ECs are cultured and maintained for multiple passages, e.g., for more than 1 or 2 months. As will be understood by one of skill in the art, the cells can be split and the cell media changed periodically during the course of culturing.

One of skill will also appreciate that the method is typically carried out with a plurality of vascular progenitor cells, to form a population of cells that, over the course of differentiation, includes increasing numbers of ECs. In some aspects, the percentage of ECs in the population of cells increases to about 50-100% over the course of differentiation, e.g., more than 65, 75, 80, 85, 90, 95 or higher percentage ECs. In some aspects, the ECs can be further separated from the population using the methods described herein and prepared for storage (e.g., in freezing media) or therapeutic application. EC cell surface markers that can be used for separation include VE-cadherin, endoglin (CD105), vWF, and Z01 (tight junction protein), though negative selection can also be used (e.g., alpha-SMA or other non-endothelial cell marker).

Methods for generating SMCs include culturing a vascular progenitor cell in a solution appropriate for SMC growth (e.g., SMC growth media) and allowing the vascular progenitor cell to form an SMC. In some aspects, the SMC growth media includes an SMC growth factor, and the cells can optionally be grown in the presence of collagen. In some aspects, the vascular progenitor cell is cultured in SMC growth media between 6 and 20 days, e.g., at least 6, 8, 10, 12 or 14 days (i.e., the course or duration of SMC differentiation). In some aspects, the vascular progenitor cell is cultured in SMC growth media for about 10-14 days. In some aspects, the SMCs are cultured and maintained for multiple passages, e.g., for more than 1 or 2 months. As will be understood by one of skill in the art, the cells can be split and the cell media changed periodically during the course of culturing.

One of skill will also appreciate that the method is typically carried out with a plurality of vascular progenitor cells, to form a population of cells that, over the course of differentiation, includes increasing numbers of SMCs. In some aspects, the percentage of SMCs in the population of cells increases to about 50-100% over the course of differentiation, e.g., more than 65, 75, 80, 85, 90, 95 or higher percentage SMCs. In some aspects, the SMCs can be further separated from the population using the methods described herein and prepared for storage (e.g., in freezing media) or therapeutic application. SMC cell surface markers that can be used for separation include alpha-SMA and calponin, though again, negative selection using an non-SMC marker can be applied.

Methods for generating a hematopoietic cell (e.g., erythrocyte (CFU-E), granulocyte-macrophage (CFU-GM), or macrophage (CFU-M)) include culturing a vascular progenitor cell in a solution or semisolid matrix appropriate for hematopoietic cell growth (e.g., hematopoietic cell growth media or matrix) and allowing the vascular progenitor cell to form a hematopoietic cell. In some aspects, the hematopoietic cell growth media includes at least one hematopoietic cell growth factor (e.g., stem cell factor (SCF), GM cell stimulating factor (GM-CSF), IL-3, EPO). In some aspects, the vascular progenitor cell is cultured in hematopoietic cell growth media between 10 and 30 days, e.g., at least 10, 12 14, 16, 18, 20, 22, or more days (i.e., the course or duration of hematopoietic cell differentiation). In some aspects, the vascular progenitor cell is cultured in hematopoietic cell growth media for about 14-21 days. In some aspects, the hematopoietic cells are cultured and maintained for multiple passages, e.g., for more than 1 or 2 months. As will be understood by one of skill in the art, the cells can be split and the cell media changed periodically during the course of culturing.

One of skill will also appreciate that the method is typically carried out with a plurality of vascular progenitor cells, to form a population of cells that, over the course of differentiation, includes increasing numbers of hematopoietic cells. In some aspects, the hematopoietic cells can be further separated from the population by picking colonies, or using the methods described herein and prepared for storage (e.g., in freezing media) or therapeutic application. CD45 can be used for separation based on cell surface markers, but morphology of the hematopoietic cell colony is another useful separation tool.

Additional information regarding cell culture techniques can be found, e.g., in Picot (2005) Human Cell Culture Protocols; Piper (1990) Cell Culture Techniques in Heart and Vessel Research; and Mather (2008) Stem Cell Culture, vol. 86, Meth. Cell Biol. Reagents and protocols for cell culture can also be found commercially, e.g., from Invitrogen, Gen-Probe, Lonza, and Clonagen, to name a few.

### V. METHODS OF TREATMENT

Further provided are methods of treatment using the differentiated cells of the invention. In some aspects, the methods of treatment comprise transplantation or engraftment of the differentiated cells into a subject for treating various EC, SMC, and hematopoietic cell deficiencies. In some aspects, the differentiated cells for treatment are derived from the subject to be treated, i.e., the cell transplant will be autologous. In some aspects, the differentiated cells for treatment are derived from a different individual, or from an induced pluripotent stem cell line. That is, the cell transplant will be allogeneic.

In some aspects, the method includes treating an EC deficiency in a subject by culturing a vascular progenitor cell in endothelial cell growth media as described herein to generate an endothelial cell, and administering the endothelial cell to the subject, thereby treating the EC deficiency. In some aspects, the administration is repeated, or combined with at least one additional therapeutic agent, e.g., to improve engraftment or ameliorate associated pain. In some aspects, the method includes culturing a progenitor stem cell in a solution comprising BMP4, bFGF, and VEGF as described herein to generate a vascular progenitor cell for further differentiation.

In some aspects, the method includes treating an SMC deficiency in a subject by culturing a vascular progenitor cell in SMC growth media as described herein to generate an SMC, and administering the SMC to the subject, thereby treating the SMC deficiency. In some aspects, the administration is repeated, or combined with at least one additional therapeutic agent, e.g., to improve engraftment or ameliorate associated pain. In some aspects, the method includes culturing a progenitor stem cell in a solution comprising BMP4, bFGF, and VEGF as described herein to generate a vascular progenitor cell for further differentiation.

In some aspects, the method includes treating a hematopoietic cell deficiency in a subject by culturing a vascular progenitor cell in hematopoietic cell growth media (or growth matrix) as described herein to generate a hematopoietic cell, and administering the hematopoietic cell to the subject, thereby treating the hematopoietic cell deficiency. In some aspects, the hematopoietic cell is selected from an erythrocyte, granulocyte-macrophage, and macrophage. In some aspects, the administration is repeated, or combined with at least one additional therapeutic agent, e.g., to improve engraftment or ameliorate associated pain. In some aspects, the method includes culturing a progenitor stem cell in a solution comprising BMP4, bFGF, and VEGF as described herein to generate a vascular progenitor cell for further differentiation.

A. Mesodermal Cell Lineage Deficiencies

The conditions that can be treated according to the present methods include deficiencies in any mesodermal cell type, e.g., endothelial (e.g., vascular endothelial), smooth muscle, hematopoietic, etc. Deficiency can result from disease, injury, or infection that affects the selected tissue.

Examples of endothelial cell deficiencies include wounds, injuries (such as an infarction), vascular diseases (e.g., atherosclerosis, coronary artery disease, hypertension, diabetes mellitus, hypercholesteremia), liver disease, or conditions that expose the endothelia to reactive oxygen species (chemical or pollution exposure). Endothelial cells can be applied to restore normal function and blood flow to affected tissues, e.g., to restore normal secretion of factors in the liver in an individual having liver disease.

SMCs can be found in large and small arteries, arterioles, veins, lymphatic vessels, urinary tract, uterus, reproductive tract, gastrointestinal tract, respiratory tract, skin, iris, and glomeruli. Thus, the methods of treatment can be applied to deficiencies, injuries, infections, and diseases in these tissues. Such deficiencies include smooth muscle condition, hepatitis, cirrhosis, lupus, atherosclerosis, etc. SMCs can be advantageously applied to repair cardiac tissue, e.g., in the case of infarct or ischemia.

Hematopoietic cell deficiencies can arise in a number of conditions. Hematopoietic cells can be advantageously applied to a subject suffering blood loss due to injury, radiation, chemotherapy, or to hematopoietic disorders or infections. Examples include anemia (e.g., sickle cell anemia), thalassemia, and macrophage deficiency.

B. Pharmaceutical Compositions and Modes of Administration

The compositions disclosed herein can be administered by any means known in the art. Typically, the differentiated cells of the invention are injected (either bolus or infusion) or otherwise applied to the affected site For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, locally, by inhalation, by injection, by infusion, by continuous infusion, by localized perfusion, via a catheter, via a lavage, in a creme, suspension, or lipid composition. Administration can be local, e.g., to the affected tissue, or systemic.

For parenteral administration in an aqueous solution, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion.

Sterile injectable solutions can be prepared by sterile filtration of the media or injection vehicle prior to incorporating the cells for injection. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more concentrated solutions for direct injection is also contemplated. DMSO can be used as solvent for rapid penetration, delivering high concentrations of the active agents to a small area.

The pharmaceutical compositions of the invention can optionally comprise growth factors or cell matrix components to support growth of the differentiated endothelial, smooth muscle, or hematopoietic cell infusion. For example, the cells can be administered in a solution of matrigel, optionally comprising, e.g., EC, SMC, or hematopoietic cell growth factors.

C. Treatment Regimes

The invention provides methods of treating, preventing, and/or ameliorating an EC, SMC, or hematopoietic cell deficiency in a subject in need thereof. The course of treatment is best determined on an individual basis depending on the particular characteristics of the subject and the type of treatment selected. Cells differentiated according to the present methods can be administered to the subject on a weekly, monthly, or any applicable basis that is therapeutically effective. The treatment can be administered alone or in combination with any other treatment disclosed herein or known in the art appropriate for EC, SMC or hematopoietic cell deficiencies, or for improving patient comfort, e.g., to reduce pain or swelling. The additional treatment can be administered simultaneously with the first treatment, at a different time, or on an entirely different therapeutic schedule (e.g., the first treatment can be daily, while the additional treatment is weekly).

### VI. KITS

Further provided are kits for generating vascular progenitor cells, or more differentiated cells of the mesodermal lineage. The kit can be designed and packaged for use in medical or research laboratories. Kits will typically comprise the reagents and optionally, disposable components needed for generation of the desired cell type.

In some aspects, the kit includes pluripotent stem cells for differentiation into vascular progenitor cells. In this case, the kit can also include culturing reagents such as bFGF, VEGF, and/or BMP4. In some aspects, the factors are packaged together in the appropriate ratios for ease of use. In some aspects, the kit includes CD34+ vascular progenitor cells for further differentiation into ECs, SMCs, or hematopoietic cells. Such kits will typically include the culturing reagents as appropriate for the particular cell type. In some aspects, the kit will include reagents for more than one differentiation program, e.g., media for endothelial cell differentiation, media for SMC differentiation, and/or media for hematopoietic cell differentiation (erythrocyte, macrophage, and granulocyte-macrophage lineages).

The cells can be frozen in appropriate buffering solution for resuspension by the user. Similarly, the necessary culture components can be packaged as concentrated stock solutions, or ready for use.

In some aspects, the kit will further comprise components for validation of the differentiation process, e.g., reagents for detecting expression of cell surface markers or other cell-type specific gene products. Accordingly, the kit can include reagents (e.g., labeled antibodies) specific for at least one of the cell surface markers selected from CD133, CD34, CD31, c-kit, KDR, Endoglin, VE-cadherin, vWF, alpha-SMA, calponin, and CD45. In some aspects, the kit will further include controls, e.g., CD34+ vascular progenitor cells, differentiated ECs, SMCs, erythrocytes, macrophages, etc., for the sake of comparison.

The kit will generally include at least one vial, test tube, flask, bottle, syringe or other container means, into which reagents and/or cells can be suitably combined or aliquoted. Kits can also include components for comparing results such as a suitable control sample, for example a positive and/or negative control. The kit can also include labware for cell culture, e.g., tissue culture plates, bottles, or tubes. Such labware can be packaged for sterile culturing techniques.

One of skill will appreciate that the kits of the invention can include any of the above components in any combination. The kit can be customizable, such that the user can request particular components on an as-needed basis.

### VII. EXAMPLES

### A. Materials and Methods

Reagents. The following antibodies were used for flow cytometry and fluorescence microscopy experiments respectively: mouse anti-human CD34-APC (130-046-703, Miltenyi), mouse anti-human CD45-FITC (130-080-202, Miltenyi), mouse anti-human CD133/2 (293C3)-PE (130-090-853, Miltenyi), mouse anti-human CD144-PE (Endoglin; 560410, BD biosciences), CD105-PE (VE-Cadherin; ab60902, Abcam), CD31-FITC (555445, Bd biosciences), CD117-PeCy7 (c-Kit; 339195, BD biosciences), VEGFR2-PE (Kdr; 560494, BD biosciences), mouse APC isotype control (555751, BD biosciences), mouse FITC isotype control (555748, BD biosciences), PeCy7 isotype control (557872, BD biosciences), PE isotype control (555749, BD biosciences), VE-Cadherin (555661, BD biosciences), Endoglin (M3527, DAKO), vWF (7356, Millipore), Calponin (Dako, M3556), αSMA (AB56994, Abcam), ZO1 (61-7300, Invitrogen).

Cell culture. Human ES cells, H1 (WA 1, WiCell), Hues 9 (available from Harvard at mcb.harvard.edu/melton/hues) and Human iPS cells CBiPS and KiPS (KIPS 4F#2, CBiPS 2F#4 (Person et al. (2008) Development 135:1525; Aasen et al. (2008) Nat. Biotechnol. 26:1276) (passage 25-45) were cultured in chemically defined hES/hiPS growth media, mTeSR on growth factor reduced matrigel (BD biosciences) coated plates. Briefly, 70-80% confluent HES/iPS cells were treated with dispase (Invitrogen) for 7 minutes at 37° C. and the colonies were dispersed to small clusters and lifted carefully using a 5 ml glass pipette, at a ratio of ˜1:4. For in vivo experiments, Human Umbilical Vein Endothelial Cells (HUVEC) were purchased from Promocell and cultivated in EBM medium supplemented with EGM singlequots (Lonza), 2% FBS, hEGF 10 μg/ml, Heparin 100 μg/ml (Sigma). iPS/ES-derived endothelial cells were cultivated in EGM-2 bullet kit (Lonza). All the cells were grown in collagen I coated plates (BD biosciences). All cell lines were maintained in an incubator (37° C., 5% CO2) with media changes every day (hES/iPS) or every second day (HUVEC).

Directed differentiation of hES/hiPS cells in chemically defined conditions. Human ES/iPS cells cultured as described above were freshly split on to matrigel-coated plates, ensuring small sub-colonies (˜300-500 cells/colony). After 24 hours of recovery in mTeSR, the cells were gently washed using DMEM/F12 (Invitrogen) and allowed to grow in chemically defined differentiation media (DMEM:F12, 15 mg/ml stem cell grade BSA (MP biomedicals), 17.5 μg/ml Human Insulin, (SAFC bioscience), 275 μg/ml Human holo transferrin (Sigma Aldrich), 20 ng/ml bFGF (Stemgent), 50 ng/ml Human VEGF-165 aa (Humanzyme), 25 ng/ml human BMP4 (Stemgent), 450 μM monothioglycerol (Sigma Aldrich), 2.25 mM each L-Glutamine and Non essential amino acids (Invitrogen), systematically designed by titration of growth factors and culture conditions. Media was changed every second day with addition of half the volume of media every other day.

RNA isolation and real time PCR (RT-PCR) analysis. Total cellular RNA was isolated using Trizol Reagent (Invitrogen) according to the manufacturer's recommendations. 2 μgs of DNAsel (Invitrogen) treated total RNA was used for cDNA synthesis using the SuperScript II Reverse Transcriptase kit for RT-PCR (Invitrogen). Real-time PCR was performed using the SYBR-Green PCR Master mix (Applied Biosystems). The levels of expression of respective genes were normalized to corresponding GAPDH values and are shown as fold change relative to the value of the control sample. All the samples were done in triplicate. The list of the primers used for real time-PCR experiments are listed in the table below.

| Gene | 5′ oligo (SEQ ID NO) | 3′ Oligo (SEQ ID NO) |
|---|---|---|
| OCT4 | GGGTTTTTGGGATTAAGTTCTTCA (1) | GCCCCCACCCTTTGTGTT (2) |
| NANOG | ACAACTGGCCGAAGAATAGCA (3) | GGTTCCCAGTCGGGTTCAC (4) |
| SOX2 | CAAAAATGGCCATGCAGGTT (5) | AGTTGGGATCGAACAAAAGCTATT (6) |
| BRACHYURY | GCCCTCTCCCTCCCCTCCACGCACAG (7) | CGGCGCCGTTGCTCACAGACCACAGG (8) |
| Tie2 | TGCCACCCTGGTTTTTACGG (9) | TTGGAAGCGATCACACATCTC (10) |
| CD31 | AACAGTGTTGACATGAAGAGCC (11) | TGTAAAACAGCACGTCATCCTT (12) |
| GATA4 | ACACCCCAATCTCGATATGTTTG (13) | GTTGCACAGATAGTGACCCGT (14) |
| HOXB4 | GTGAGCACGGTAAACCCCAAT (15) | CGAGCGGATCTTGGTGTTG (16) |
| CD34 | CCTAAGTGACATCAAGGCAGAA (17) | GCAAGGAGCAGGGAGCATA (18) |
| ANG1 | GGGGGAGGTTGGACTGTAAT (19) | AGGGCACATTTGCACATACA (20) |
| ANG2 | GGATCTGGGGAGAGAGGAAC (21) | CTCTGCACCGAGTCATCGTA (22) |
| HOXB4 | GTGAGCACGGTAAACCCCAAT (23) | CGAGCGGATCTTGGTGTTG (24) |
| GATA1 | AGAAGCGCCTGATTGTCAGTA (25) | AGAGACTTGGGTTGTCCAGAA (26) |
| GATA2 | GGCCCACTCTCTGTGTACC (27) | CATCTTCATGCTCTCCGTCAG (28) |
| RUNX1 | AGAACCTCGAAGACATCGGC (29) | GGCTGAGGGTTAAAGGCAGTG (30) |
| CXCR4 | CACCGCATCTGGAGAACCA (31) | GCCCATTTCCTCGGTGTAGTT (32) |
| LMO2 | GGACCCTTCAGAGGAACCAGT (33) | GGCCCAGTTTGTAGTAGAGGC (34) |
| SCL | CAAAGTTGTGCGGCGTATCTT (35) | TCATTCTTGCTGAGCTTCTTGTC (36) |
| TEL | AAACTTCATCCGATGGGAGGA (37) | CGCAGGGCTCTGGACATTTT (38) |
| FOXA1 | CCAAGGCCGCCTTACTCCTACA (39) | CGCAGATGAAGACGCTTGGAGA (40) |
| TEL | AAACTTCATCCGATGGGAGGA (41) | CGCAGGGCTCTGGACATTTT (42) |
| ETV2 | CCGACGGCGATACCTACTG (43) | GTTCGGAGCAAACGGTGAG (44) |
| FOXC1 | AAGATCACGCTCAATGGGATTT (45) | ACAGGTTGTGACGGATGCTG (46) |
| TUBB3 | CCTGGAACCCGGAACCAT (47) | AGGCCTGAAGAGATGTCCAAAG (48) |
| ASMA | CAGGGCTGTTTTCCCATCCAT (49) | GCCATGTTCTATCGGGTACTTC (50) |
| CALPONIN | GAGTCAACCCAAAATTGGCAC (51) | GGACTGCACCTGTGTATGGT (52) |
| SM-MHC | GGACGACCTGGTTGTTGATT (53) | GTAGCTGCTTGATGGCTTCC (54) |
| CALDESMON | AACAACCTGAAAGCCAGGAGG (55) | GCTGCTTGTTACGTTTCTGC (56) |
| SMα-22 | CGCGAAGTGCAGTCCAAAATCG (57) | GGGCTGGTTCTTCTTCAATGGGG (58) |
| VE-CADHERIN | GACCGGGAGAATATCTCAGAGT (59) | CATTGAACAACCGATGCGTGA (60) |
| ENDOGLIN | CCCACAAGTCTTGCAGAAACA (61) | CTGGCTAGTGGTATATGTCACCT (62) |
| vWF | ATGTTGTGGGAGATGTTTGC (63) | GCAGATAAGAGCTCAGCCTT (64) |
| GAPDH | GGACTCATGACCACAGTCCATGCC (65) | TCAGGGATGACCTTGCCCACAG (66) |

Flow cytometry. Human ES/iPS cells undergoing directed differentiation or endothelial cells derived from Human ES/iPS cells were harvested using TrypLE (Invitrogen), washed once with PBS and further incubated with the corresponding antibodies in the presence of FACS blocking buffer (1× PBS/10% FCS) for 1 hour on ice in the absence of light. After incubation, cells were washed thrice with 1 ml of FACS blocking buffer and resuspended in a total volume of 200 μl prior to analysis. A minimum of 10,000 cells in the living population was analyzed. Percentages are presented after subtracting isotype background and referring to the total living population analyzed. Results are representative of at least two independent experiments with a minimum of two technical replicates per experiment

Magnetic cell sorting (MACS). After 8 days of differentiation, CD34+ cells were enriched using anti-CD34 conjugated magnetic beads (Miltenyi biotec) according to the manufacturer's instructions with slight modifications. Briefly, up to 10⁹ cells were incubated with constant mixing at 4° C. with 100 μl of the corresponding magnetic beads in the presence of 100 μl of Fc-blocking solution in a total volume of 500 μl FACS blocking buffer. After 1 hour, cells were sorted by two consecutive rounds of column separation in order to increase purity by applying MACS separation magnets. Briefly, cells were passed through the first MS separation column allowing binding of labeled cells. Non-labeled cells were washed thoroughly with 3 ml FACS blocking buffer prior to elution of the labeled fraction. Eluted labeled cells were then subjected to a second purification step as described above.

Differentiation conditions for CD34+ cells to endothelial cells. Briefly, CD34+ cells isolated by MACS after 8 days of differentiation were plated in a well with EGM2 (Lonza) on Collagen 1 coated plates (50,000 cells/well of a 12 well plate). The endothelial cell media has EGF and can have serum. After 5-8 days of culturing, upon reaching 90% confluence, cells were split 1:4, using TrypLE (Invitrogen). The cells were cultured for at least 8 passages. The mature endothelial cells could be frozen and thawed with >90% recovery and kept in culture for at least 8 passages without showing signs of senescence.

Differentiation conditions for CD34+ cells to smooth muscle cells. Briefly, CD34+ cells isolated by MACS after 8 days of differentiation were plated in a well with SmGM (Lonza) on Collagen 1 coated plates (50,000 cells/well of a 12 well plate). The smooth muscle cell growth media can have EGF, FGF, and serum. After 5-8 days of culturing, upon reaching 90% confluence, cells were split 1:4, using TrypLE (Invitrogen). The cells were cultured for at least 8 passages.

Hematopoietic colony forming assays. Hematopoietic clonogenic assays were performed in 35-mm low adherent plastic dishes (Stem Cell Technologies, Vancouver, BC, Canada) using 1.1 ml/dish of methylcellulose semisolid medium (MethoCult H4434 classic, Stem Cell Technologies) according to the manufacturer's instructions. Briefly, enriched CD34+ cells derived from indicated human/ES/iPS cells were sorted and immediately plated at a concentration of 1×10⁴ cells/ml. All assays were performed in duplicate. Colony-forming units (CFU) were identified after 14 to 21 days of incubation according to their colony morphology as erythroid (CFU-E), granulocyte-macrophage (CFU-GM), and macrophage (CFU-M).

Fluorescence microscopy. Briefly, cells were washed once with PBS, fixed using 4% PFA in 1× PBS. After fixation, cells were blocked and permeabilized for 1 hr at 37° C. with 5% BSA/5% appropriate serum/1× PBS with 0.1% Triton X100. Subsequently, cells were incubated with the indicated primary antibody overnight at 4° C. The cells were then washed thrice with 1× PBS and incubated for 1 hr at 37° C. with the respective secondary antibodies and 20 minutes with DAPI (0.5 μg/ml in PBS). Cells were washed thrice with 1× PBS before analysis.

Acetylated LDL uptake assay and vascular tube-like structure formation assay. In brief, 80% confluent endothelial cells derived from human ES/iPS cells were incubated with 10 μg/ml Dil-Ac-LDL (L23380, Molecular Probes) for 3 hours in DMEM/F12. The cells were washed 3 times with PBS, dissociated using TrypLE and analyzed by flow cytometry. To assess the formation of capillary structures, 1×10⁵ endothelial cells were incubated on Matrigel (BD biosciences) in complete EGM-2 for 24 h.

Animals. Animal experiments were performed in accordance with institutional guidelines and national legislation. NOD.Cg-PrkdescidIl2rgtmlWjl/SzJ mice (or NOD-Scid IL2Rγnull abbreviated as NSG; aged 7 weeks; weight 20 g were purchased from Charles River Laboratories, housed in air-flow racks on a restricted access area and maintained on a 12-h light/dark cycle at a constant temperature (22±1° C.).

Matrigel plug assay. Anesthesia was induced by using a mixture of Xylazine (Rompun® 2%, Bayer) at 10 mg/kg and Ketamine (Imalgene1000, Merial) at 100 mg/kg in NaCl at 0.9% i.p injected at a dose of 10 mL/kg. The animal backs were shaved, swabbed with Hexomedine®. HUVECs, HUES9-derived endothelial, and non-endothelial cells were harvested using TrypLE (Invitrogen) and resuspended in 500 μl of matrigel (Matrigel basement membrane matrix from BD Biosciences adjusted to 9.8 mg/ml PBS) supplemented with 150 ng of bFGF. Cell and no cell containing matrigel solutions were then injected subcutaneously in the back of mice, carefully positioning the needle between the epidermis and the muscle layer. Thirteen days later, mice were sacrificed and the matrigel plugs were removed by a wide excision of the back skin, including the connective tissues (skin and all muscle layers).

Tissue processing and analysis. For immunohistochemistry (IHC), in situ hybridization (ISH) or immunofluorescence (IF) analysis, cell-containing implants with associated connective tissues were fixed with Accustain® (SIGMA), dehydrated through an ethanol series and then processed for paraffin embedding. Slides from paraffin-embedded samples were stained with appropriate antibodies or probes. For IHC, slides were stained with an anti human-CD31 monoclonal antibody and then incubated with a biotin-labeled secondary antibody followed by incubation with streptavidin-HRP (Ventana Roche). For ISH, slides were hybridized according to the manufacturer's protocol with an Alu probe (a fluorescein-labelled oligonucleotide probe that has an affinity to human ALU specific sequences, Ventana Roche) and then labeled with the. ISH iView Blue Detection kit (an indirect biotin/streptavidin/alkalin phosphatase system for detecting fluorescein-labeled probes, Ventana Roche). For IHC and ISH, images were then captured with a camera mounted on a light microscope (Nikon E-800). For immunofluorescence assays, slides were stained with rhodamine-labeled Ulex Europaeus Agglutinin I (UEA I, a marker for human endothelial cells from Vector Laboratories; Burlingame, Calif.) and images were captured with a confocal microscope (Zeiss, LSM 510).

Statistical evaluation. Statistical analyses of all endpoints were performed by using standard unpaired Student t test (one-tailed, 95% confidence intervals) using SPSS/PC+ statistics 11.0 software (SPSS Inc., Chicago, Ill.). All data are presented as mean±standard deviation and represent a minimum of two independent experiments with at least two technical duplicates.

### B. Example 2

### Generation of Hemagioblast-Like Cells From Pluripotent Stem Cells

Normal embryonic development of hemangioblast-like cells is a spatially and temporally well-orchestrated process carried out by a partially overlapping interplay of different signaling pathways, including TGFβ, Wnt, Notch, bFGF, Activin-A and VEGF. Activation of the “hemangioblast program” results in the expression of essential transcription factors (TFs) including SCL, LMO2, GATA1, GATA2 and RUNX1 both in vitro and in vivo. These cells can give rise to endothelial and smooth muscle cells (ECs and SMCs respectively), the so-called angioblast population, and/or to endothelial, smooth muscle and hematopoietic cells, the hemangioblast population.

We sought to establish a robust, defined differentiation process suitable for molecular studies and translation into the clinic. In order to limit confounding effects, e.g., due to the presence of viral vectors for exogenous gene expression, we initially selected Cord Blood-derived induced pluripotent cells (CBiPS) cells reprogrammed by Oct4 and Sox2 transduction as described in Giorgetti et al. (2009) Cell Stem Cell 5:353-57. CD34, an early marker for hemangioblast development expressed in human multipotent progenitor cells, was used to identify hem angioblast-like cells.

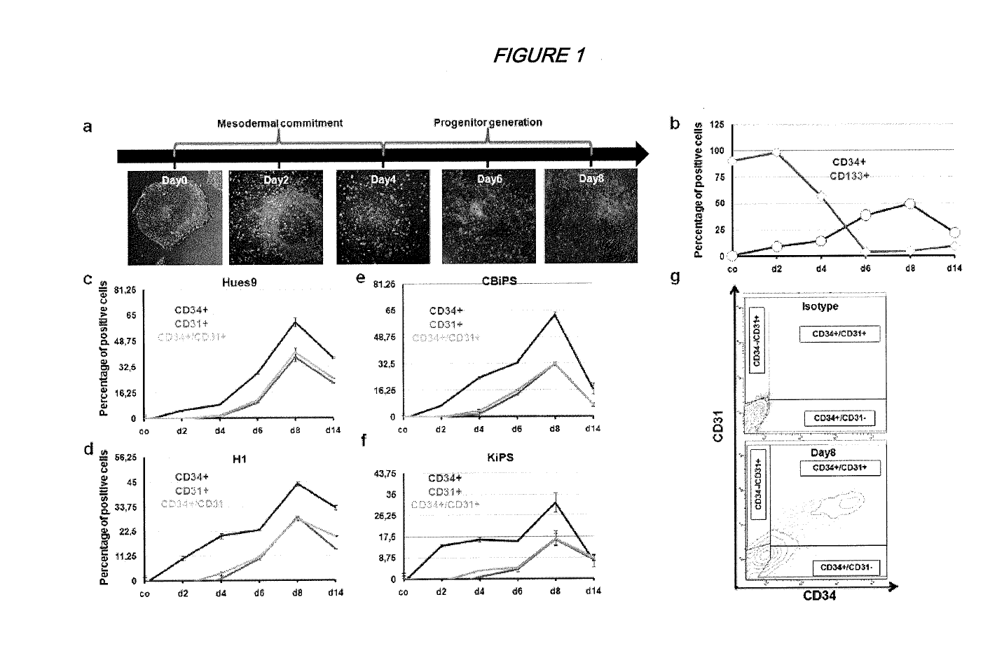

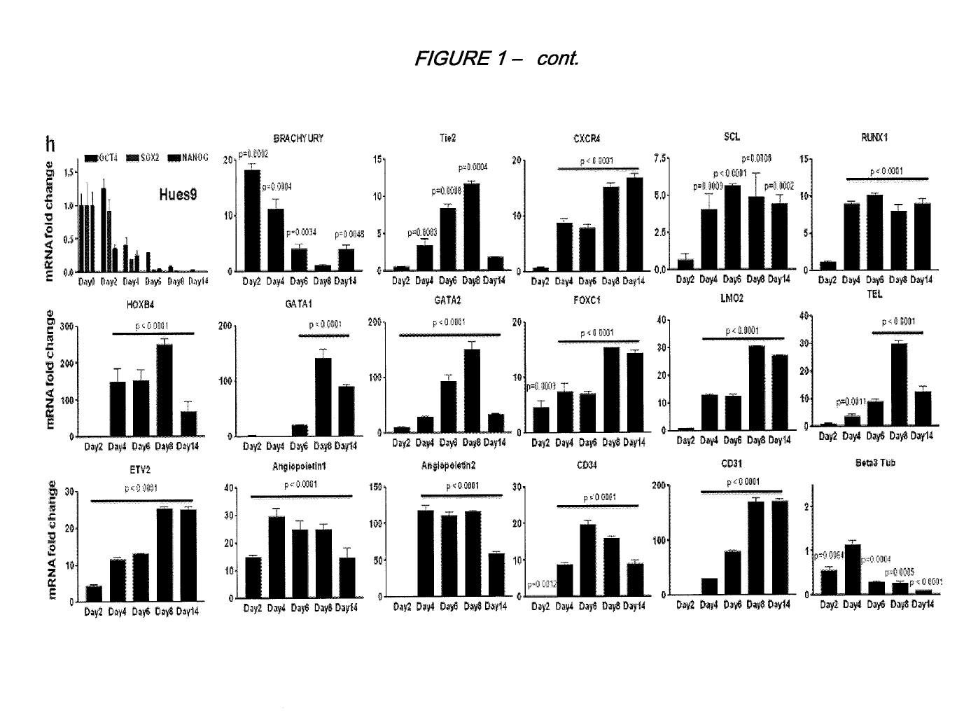

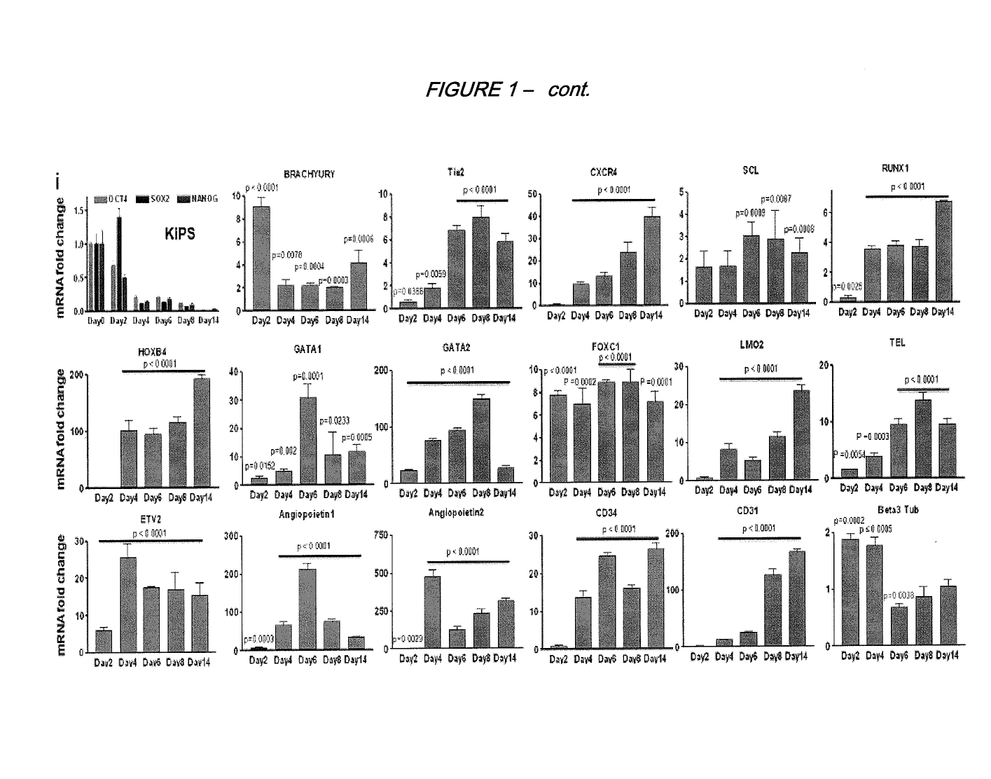

We cultured the CBiPS in a chemically defined media containing 25 ng/ml human BMP4, 20 ng/ml bFGF and 50 ng/ml human VEGF to differentiate the pluripotent stem cells to hemangioblast-like cells. Interestingly, we saw efficient generation of CD34+ cells with either with or without 3 ng/ml Activin-A. Thus, we omitted Activin-A from the culture conditions. In addition, we elected to omit feeder cells, again, to reduce the potential for confounding factors, and also to better facilitate translation into the clinic by reducing the presence of contaminants. Using these conditions, we observed efficient differentiation from progenitor stem cell (CBiPS) to hemangioblast-like cells within 8 days (FIG. 1a).

The differentiating cells were observed every second day (FIG. 1a). By day 2, we observed loss of the typical iPS/ES (embryonic stem cell) compact colony morphology in favor of a more spread-out colony shape, indicative of differentiation (FIG. 1a). We also compared the relative expression of the stem cell marker CD133 and the early hematopoietic/hemangioblast marker CD34 by flow cytometry. As depicted in FIG. 1, expression of CD133 significantly dropped over the first 4-6 days of in vitro differentiation (FIG. 1b). A significant upregulation of CD34 paralleled the decrease in CD133 expression. Interestingly, we did not find any significant population bearing both CD133 and CD34; the markers were mutually exclusive during the differentiation process (FIG. 1b).

We next evaluated the robustness of the present differentiation process on multiple human iPS (hiPS) and human ES (hES) cells. We routinely observed between 30-65% CD34+ in all hES and hiPS cell lines tested (FIG. 1c-g), with the majority falling in the range of 45-65%. Although we generally observed a lower progenitor generation potential with Keratinocyte-derived iPS (KiPS), they consistently yielded more than 30% CD34+ cells (FIG. 1f). To date we have tested a total of 12 different iPS lines from different developmental origins with similar results, thus demonstrating the robustness of our method regardless of the cell line employed for differentiation.

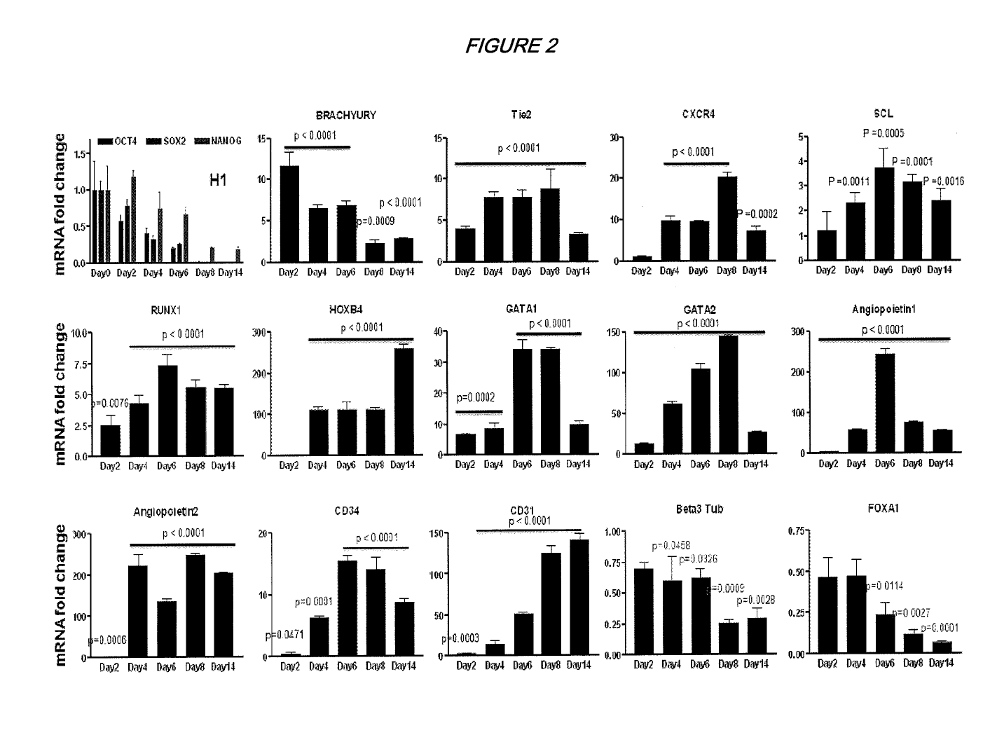

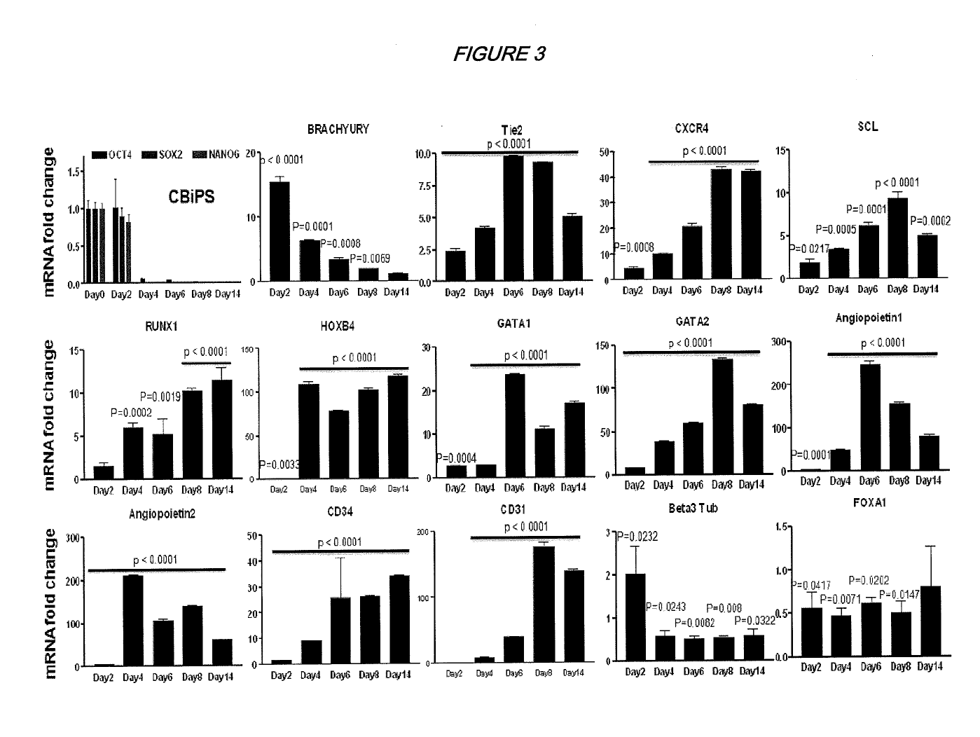

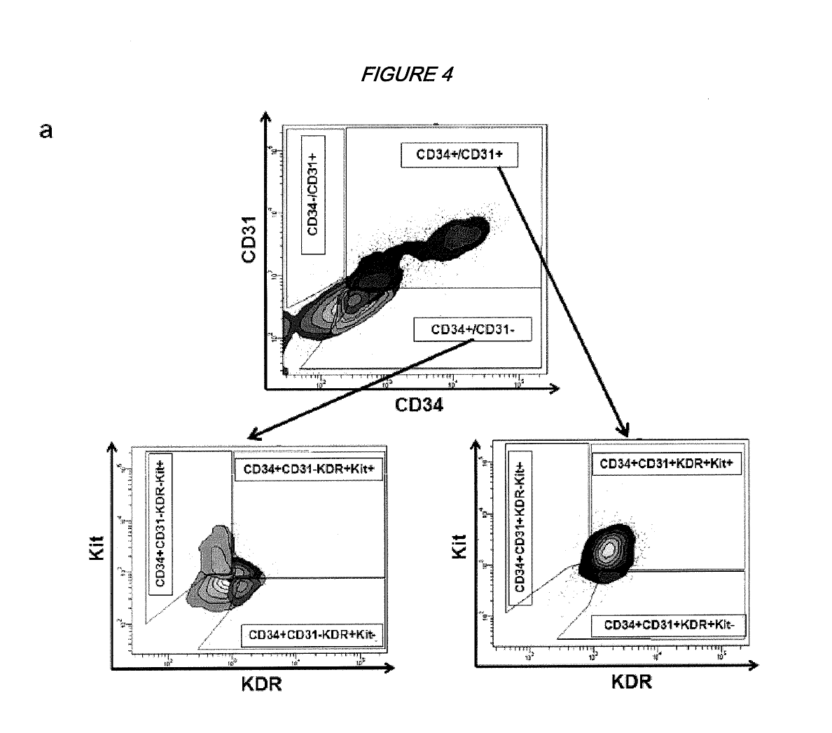

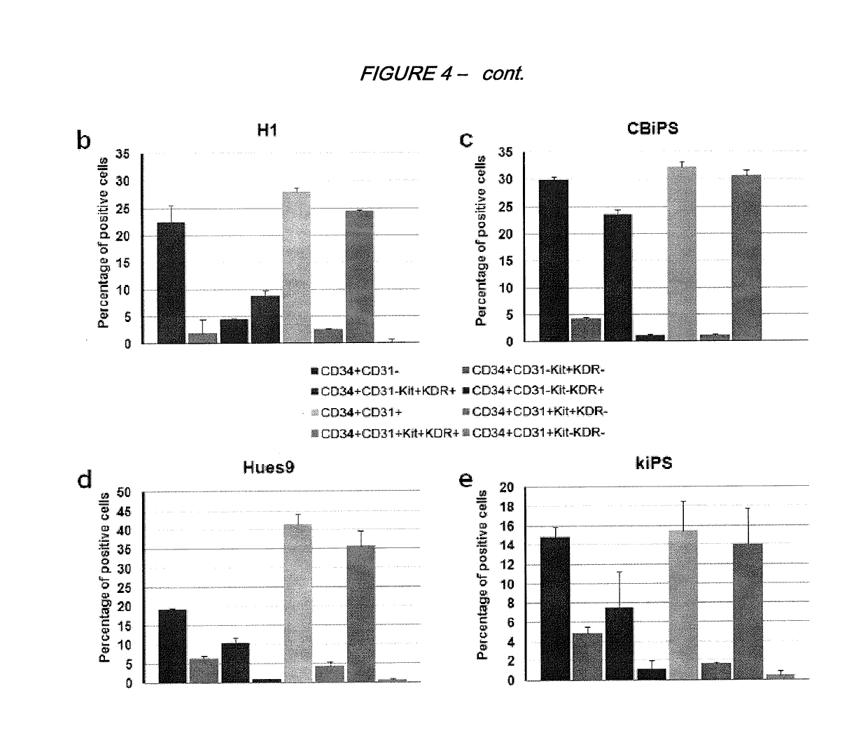

The peak of CD34+ cells occurred by day 8 in every cell line analyzed. Expression of the hemangioblast-related and endothelial marker CD31 paralleled the peak expression of CD34. Interestingly, most of the CD31+ cells were CD31+CD34+, such that the double positive cells represented up to 50% of the total CD34+ cells and up to 45% of the total of number of cells (FIG. 1c-g and FIG. 4). Importantly, we observed rapid downregulation of the pluripotency markers Sox2, Oct4, KLF4 and c-Myc by day 2, indicative of differentiation (FIG. 1h-i, FIG. 2, FIG. 3). By day 4, expression of pluripotency markers was barely detectable (FIG. 1h-i, FIG. 2, FIG. 3).

Expression of Brachyury, one of the earliest mesodermal markers, was strongly upregulated by day 2, and subsequently attenuated during the course of differentiation (FIG. 1h-i, FIG. 2, FIG. 3). Expression of the early hemangioblast markers Tie2, SCL, CXCR4 and Runx1 started at day 2 and significantly increased from day 4 to 8 (FIG. 1h-i, FIG. 2, FIG. 3). In addition, expression of hemangioblast-related transcription factors such as HoxB4, GATA1, GATA2, LMO2, Tel and Etv2 significantly increased over the basal levels observed in non-differentiating conditions (FIG. 1h-i, FIG. 2, FIG. 3).

The expression level of the non-hemangioblast related marker βIII-Tubulin, a neuronal and ectoderm marker was determined to ensure specificity of the differentiation pathway. As expected, expression of βIII-Tubulin was significantly downregulated by day 4 onwards (FIG. 1h-i, FIG. 2, FIG. 3).

Thus, the present method provides for the robust directed differentiation of hES/hiPS towards cell populations bearing both endothelial and hematopoietic markers, regardless of the cell line used. Furthermore, the observed progression of defined marker expression faithfully resembled embryonic development. Thus, the methods described herein represent an excellent tool for in vitro developmental and signaling studies, as well as for generating therapeutic cell populations for transplant.

### C. Example 3

### Characterization of Hemangioblast-Like Cells

We focused on the day 8 population for further sorting and analysis of terminal differentiation of these hemangioblast-like progenitors derived from hES and hiPS cells. We first fully characterized all different subpopulations observed at day 8 in terms of mesodermal-related cell surface markers KDR, c-Kit, CD34 and CD31 by flow cytometry. FIG. 4a shows the representative gating strategy employed. Briefly, we gated the living population and showed the relative expression of CD34 and CD31 on contour plots in order to obtain additional visual information regarding the density of the populations. We initially defined CD34−CD31−, CD34+CD31− and CD34+CD31+ populations. Each of those populations was further gated and represented in terms of relative expression for KDR and c-Kit (FIG. 4a, lower panels). As previously discussed, essentially all CD31+ cells were also CD34+, and no significant CD34−CD31+ population was observed in any of the lines.

In order to directly estimate the relative percentages of each four-marker subpopulation, we calculated the percentages versus the initial living population, not referring to the gated CD34−CD31−, CD34+CD31− or CD34−CD31+ populations (FIG. 4a-e). Almost every CD34+CD31+ cell co-expressed KDR and c-Kit. Thus, essentially all CD31+ cells are CD34+CD31+KDR+c-Kit+ cells, which comprise up to 35% of the total living population. This represents the highest percentages described to date. Moreover, CD34+CD31− cells were primarily c-Kit+, with no clear pattern of KDR expression (FIG. 4a-e). Interestingly, all CD34+CD31+ populations showed significantly higher fluorescence intensities for CD34 compared to CD34+CD31−, thus representing a CD34^{hi}CD31+ isolated population (FIG. 4a).

### D. Example 4

### Differentiation of Hemangioblast-Like Cells into Endothelial, Smooth Muscle, and Hematopoietic Cells

CD34+ and CD31+ populations have similar potential to differentiate into endothelial cells (EC) and smooth muscle cells (SMC). We next investigated the multilineage potential of the sorted CD34+ populations by enriching the CD34+ cells.

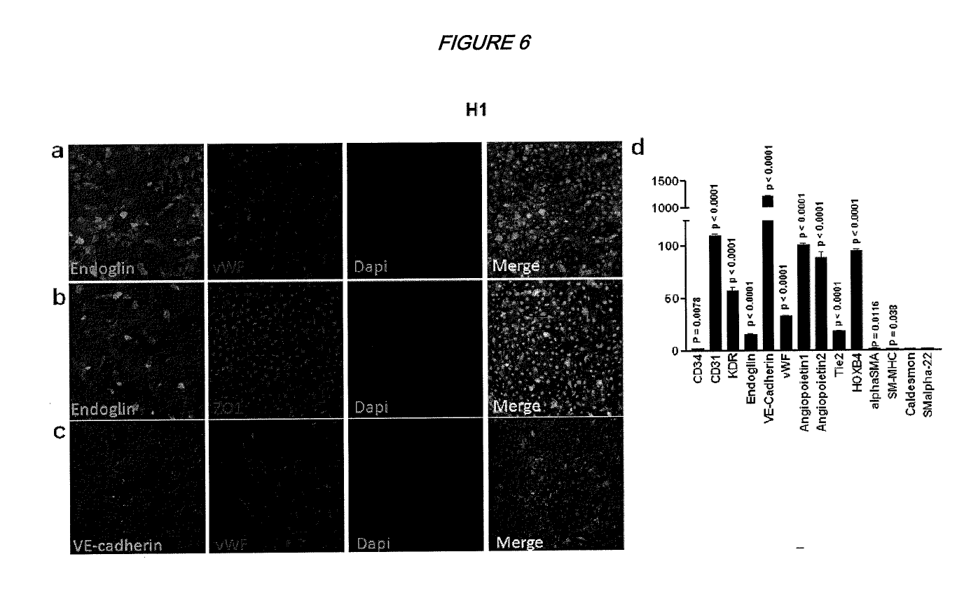

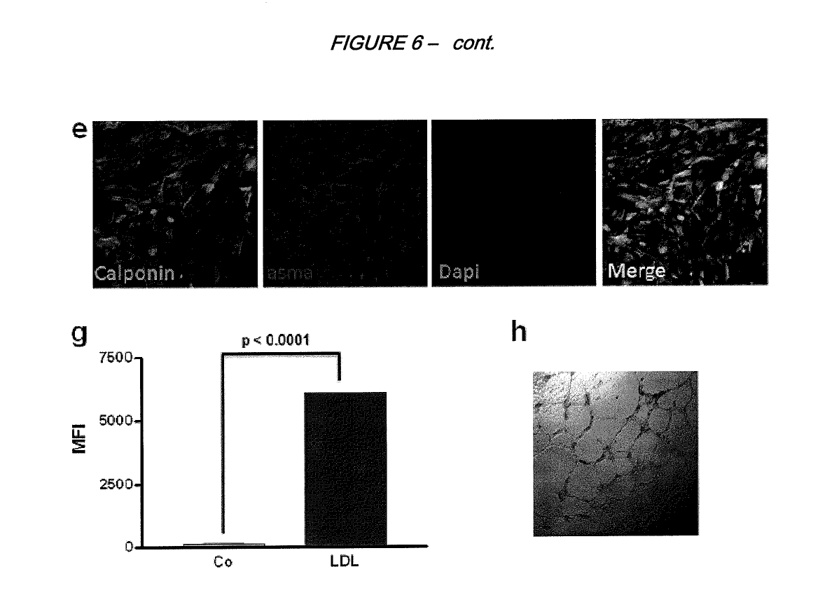

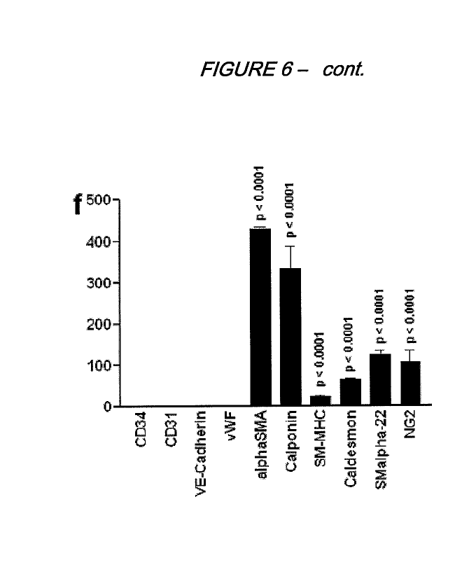

Directed differentiation of CD34+ cells toward the endothelial lineage over 10-14 days robustly gave rise to homogenous populations of up to 100% VE-Cadherin and Endoglin positive cells (FIG. 5a-e and FIG. 6-8). Endoglin is a surface marker expressed on actively proliferating endothelial cells. Accordingly, when cells reached confluency, surface expression of Endoglin was barely detectable and Endoglin could be readily detected in the cytoplasm (FIG. 5a-e, FIG. 6-8). VE-Cadherin is a mediator of cell-cell contact, and is characteristic of the endothelial lineage. However, VE-Cadherin is not specific for terminally differentiated endothelial cells, as it has been observed in late mesodermal progenitors. Thus, to further confirm the mature identity of the differentiated cells, we included an extensive panel of mature endothelial markers such as vWF and Z01 in combination with VE-Cadherin and Endoglin. As shown in FIG. 5, almost every cell co-expressed every other endothelial marker studied, thus demonstrating the endothelial nature of the newly generated cells (FIG. 5c-e and FIG. 6-8).

To further validate the endothelial identity of the differentiated cells, we analyzed mRNA expression of specific endothelial and smooth muscle markers. qPCR analysis showed significant downregulation of the progenitor marker CD34. Every endothelial marker analyzed was significantly upregulated compared to basal levels observed in non-differentiation conditions (FIG. 5f). In addition, we did not observe upregulation of almost any of the smooth muscle markers analyzed (FIG. 5f and FIG. 6-8).

We next exposed the CD34+ hemangioblast-like cells to directed differentiation conditions for the smooth muscle cell lineage. This resulted in specific and efficient differentiation to almost 100% smooth muscle cells, as identified by co-expression of the SMC-specific markers Calponin and α-Smooth Muscle Actin (αSMA) (FIG. 5g and FIG. 6-8). mRNA analysis confirmed this results by showing significant upregulation of smooth muscle specific markers, while no endothelial marker upregulation was observed (FIG. 5g-h and FIG. 6-8).

Thus, the present differentiation process can not only generate massive amounts of CD34+ cells in a short time-frame, but the CD34+ cells can efficiently differentiate into the endothelial and smooth muscle lineages.

We also tested the ability of the CD34+ hemangioblast-like cells to generate hematopoietic cells. Hematopoietic differentiation was restricted to the presence of erythroid, granulocyte-macrophage, and macrophage lineages. This distribution resembles that of yolk sac blood islands, in which most of the hematopoietic system is purely comprised of macrophages and erythrocytes during early embryonic development (FIG. 9).

### E. Example 5

### Characterization of Differentiated Endothelial Cells

Endothelial cells are characterized by high Low-Density-Lipoprotein (LDL) uptake, thus, we examined the differentiated endothelial cells functionally by measuring acetylated-LDL uptake. The CD34+ derived endothelial cells showed significantly higher rates of LDL uptake than the respective negative controls (FIGS. 5i, j and FIG. 6-8). Endothelial cells are also known to spontaneously aggregate into capillary-like structures when cultured on matrigel, an example of “in vitro” vessel formation capacity. The CD34+ derived endothelial cells were also able to efficiently aggregate into vessel-like structures in vitro (FIG. 5k and FIG. 6-8).

A major drawback to previous hiPS differentiation protocols is that the hemangioblast-like cells generated thereby undergo early senescence. We did not observe senescence of the cells differentiated according to the present methods (FIG. 5l-p). The differentiated endothelial cells could be amplified in culture for at least 8 passages. Moreover, freezing and thawing of the cells did not negatively impact the angiogenic properties (FIG. 5l-p and FIG. 6-8).

We also tested the ability of the differentiated endothelial cells to form functional vessels in vivo. To do so, we injected matrigel plugs, with or without cells, subcutaneously into mice and allowed the cells to form vessels in the plug and surrounding tissue. After 13 days, the mice were sacrificed, and the tissues processed for analysis. The results show that indeed, the differentiated endothelial cells were able to form functional vessels in vivo and connect to the pre-existing vasculature and blood circulation even after freezing and thawing steps (FIG. 5l-p). Thus, cells differentiated according to the present methods can be banked and stored, facilitating rapid clinical application when needed.

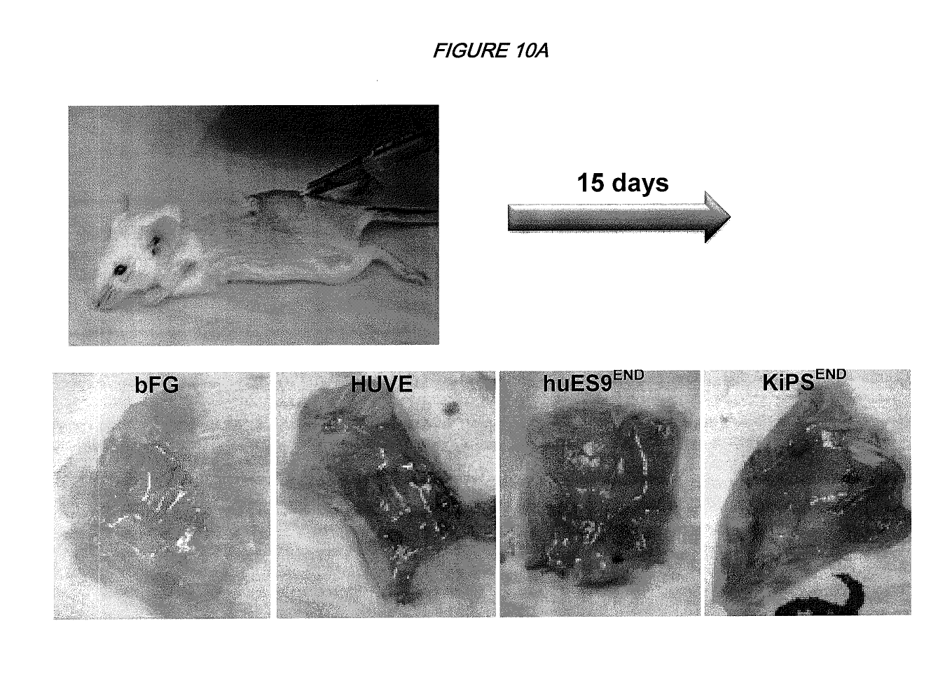

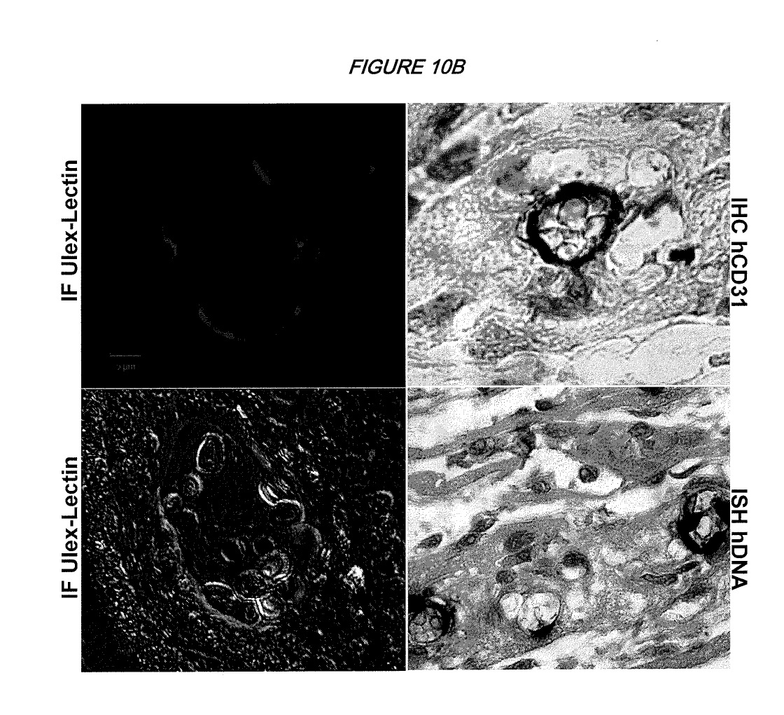

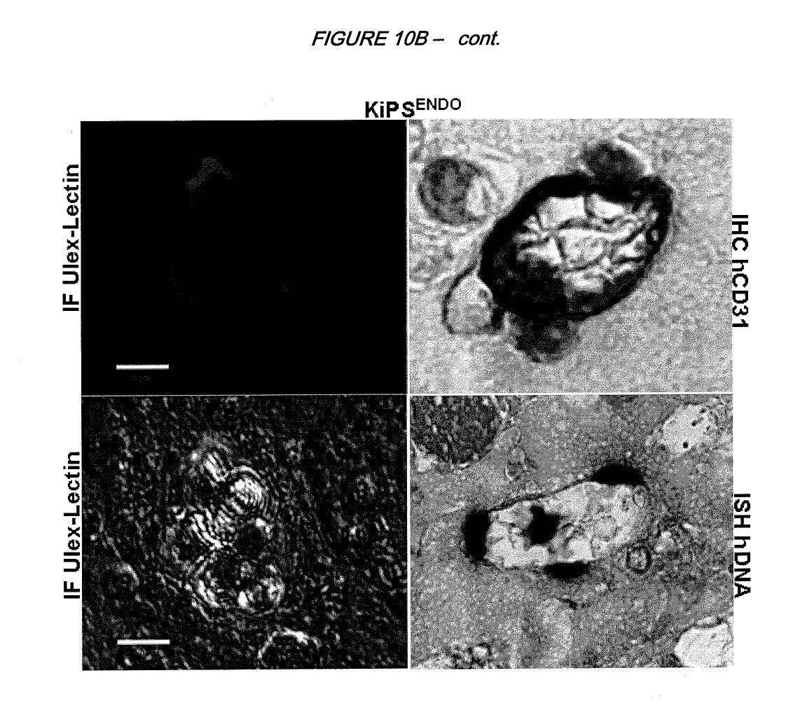

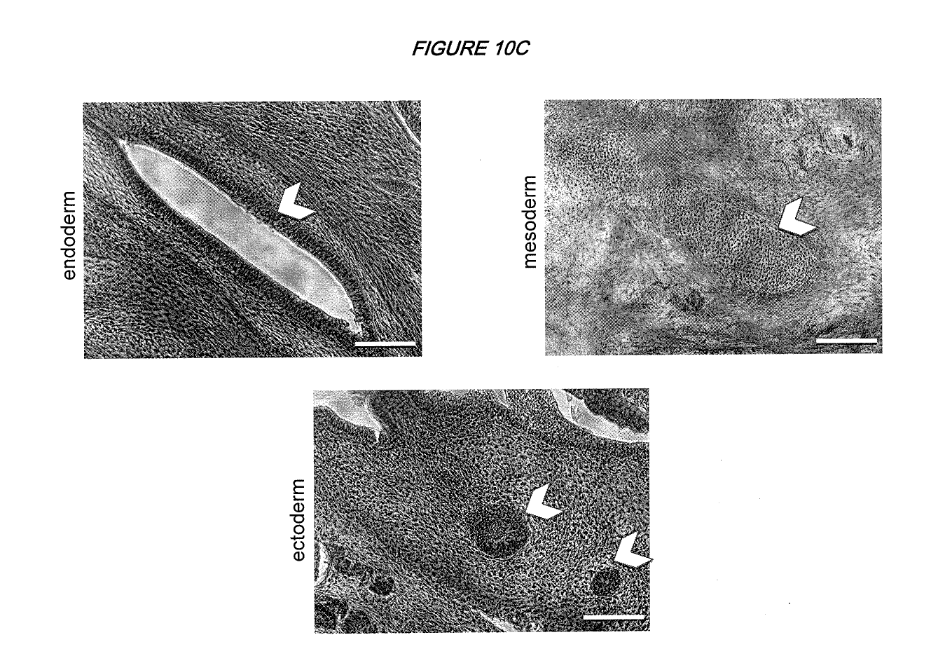

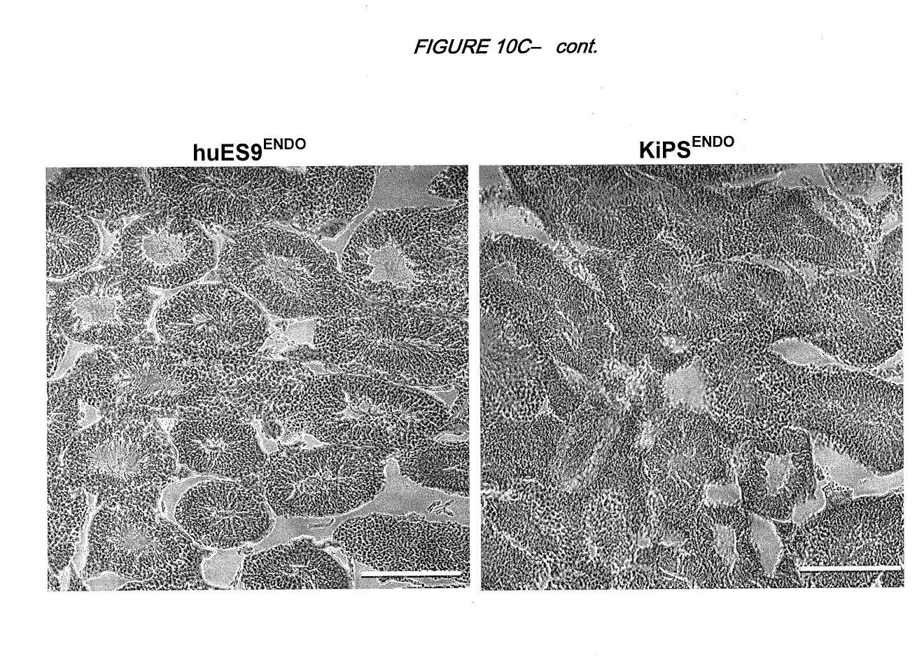

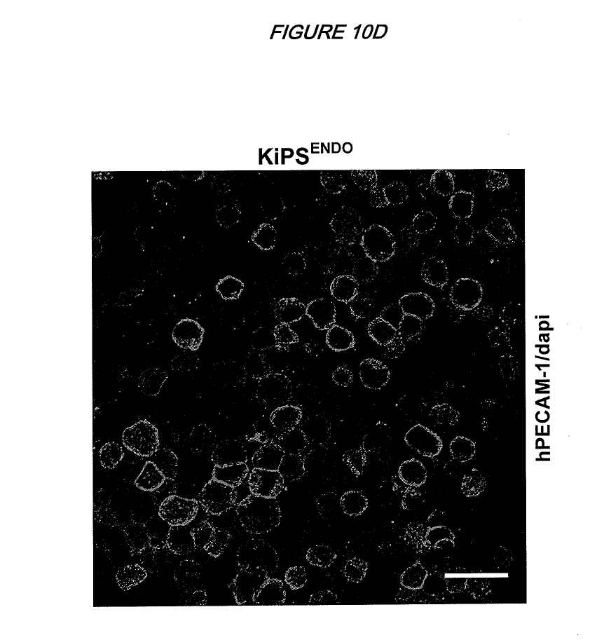

These results were confirmed in an additional in vivo test using matrigel plugs. Endothelial cells generated as described above were introduced into the plugs and injected into mice. Fifteen days after injection, the matrigel plugs were extracted and analyzed. Again, the plugs showed significant blood circulation, demonstrating connection with existing vasculature (FIG. 10a). In situ hybridization shows vessel formation (FIG. 10b, left panels). ALU+ and anti-human CD31 staining (FIG. 10b right panels) show that the cells are indeed the human differentiated endothelial cells. FIGS. 10c and 10d show that differentiated endothelial cells do not form teratomas in vivo, unlike the less differentiated iPSC controls.

These results show that directed differentiation of human iPS (hiPS) and human ES (hES) cells can be used to generate functional, differentiated cells that are safe for administration.

The foregoing discussion of the invention is for the purposes of illustration and description, and is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. All publications, patents, patent applications, Genbank numbers, and websites cited herein are hereby incorporated by reference in their entireties for all purposes.

1. A method for generating a vascular progenitor cell comprising culturing a pluripotent stem cell in a solution comprising BMP4, bFGF, and VEGF, thereby generating a vascular progenitor cell.

2. The method of claim 1, wherein said vascular progenitor cell is CD34+.

3. The method of claim 1, wherein said vascular progenitor cell can further differentiate into an endothelial cell or a smooth muscle cell.

4. The method of claim 1, wherein said pluripotent stem cell is derived from an embryonic stem cell or an induced pluripotent cell.

5. The method of claim 1, wherein said culturing is carried out in the absence of feeder cells, serum, or Activin-A.

6.-7. (canceled)

8. The method of claim 1, wherein said culturing is carried out for at least 4 days but less than 10 days.

9.-10. (canceled)

11. The method of claim 1, wherein said method is carried out with a plurality of pluripotent stem cells, to form a population of cells comprising a plurality of vascular progenitor cells.

12. The method of claim 11, further comprising separating the vascular progenitor cells from the population of cells.

13. The method of claim 12, further comprising culturing the separated vascular progenitor cells with endothelial cell growth media to generate endothelial cells.

14. The method of claim 13, wherein said endothelial cell growth media comprises collagen.

15. The method of claim 13, wherein said endothelial growth media does not comprise feeder cells.

16. (canceled)

17. The method of claim 12, further comprising culturing the separated vascular progenitor cells with smooth muscle cell growth media to generate smooth muscle cells.

18. The method of claim 17, wherein said smooth muscle cell growth media comprises collagen.

19. The method of claim 17, wherein said smooth muscle cell growth media does not comprise feeder cells.

20. (canceled)

21. A method for generating an endothelial cell comprising:
(i) culturing a pluripotent stem cell in a solution comprising BMP4, bFGF, and VEGF, thereby generating a vascular progenitor cell; and
(ii) culturing the vascular progenitor cell in endothelial cell growth media, thereby generating an endothelial cell.

22. (canceled)

23. The method of claim 21, wherein the endothelial cell growth media comprises endothelial growth factor.

24. (canceled)

25. An isolated endothelial cell generated according to the method of claim 21.

26. A method for generating a smooth muscle cell comprising:
(i) culturing a pluripotent stem cell in a solution comprising BMP4, bFGF, and VEGF, thereby generating a vascular progenitor cell; and
(ii) culturing the vascular progenitor cell in smooth muscle cell growth media, thereby generating a smooth muscle cell.

27.-28. (canceled)

29. An isolated smooth muscle cell generated according to the method of claim 26.

30.-31. (canceled)
